(19) United States
(12) Patent Application Publication
(10) Pub. No.: US 2021/0398623 A1
(43) Pub. Date: Dec. 23, 2021

(54) SECURE PRODUCTION OF DYNAMICALLY-ALTERABLE INSTRUCTIONS

(71) Applicant: Collaborative Network 4 Clinical Excellence, Inc., Denver, CO (US)

(72) Inventor: Lukasz Kowalczyk, Denver, CO (US)

(21) Appl. No.: 17/224,035

(22) Filed: Apr. 6, 2021

Related U.S. Application Data
(60) Provisional application No. 63/005,812, filed on Apr. 6, 2020.

(51) Int. Cl. G16H 10/60; G16H 10/20; G16H 70/00; G06F 16/93

(52) U.S. Cl. CPC G16H 10/60; G06F 16/93; G16H 70/00; G16H 10/20

(57) ABSTRACT

Methods and systems are provided for a dynamically-alterable set of instructions personalized to a patient. The set of instructions may update based on patient history and/or medical professional approval. The set of instructions may keep certain portions of patient data confidential, and may dynamically trigger different workflows based on patient response.

### CROSS REFERENCE TO RELATED APPLICATIONS

The present document is a U.S. Non-Provisional patent application that claims benefit to U.S. Provisional Patent Application Ser. Number 63/005,812, filed on Apr. 6, 2020, which is herein incorporated by reference in its entirety.

### FIELD

The present disclosure is generally directed to systems devices and methods for generating and providing adaptable instructions to a patient for a procedure.

### BACKGROUND

Digital operations in the healthcare industry are critical to providing efficient and accurate medical treatment, and various technical improvements are needed. It is with these observations in mind among others that various aspects of the present disclosure were conceived and developed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is an image of one embodiment of a system for providing a set of dynamic instructions to a patient.

FIG. 2 is an image of a scheduler interface login screen.

FIG. 3 is an image of a home screen of the scheduler interface described herein.

FIG. 4 is an image of a home screen of the scheduler interface described herein.

FIG. 5 is an image illustrating the to-do list and the urgent/action required list as described herein.

FIG. 6 is an image illustrating the to-do list and the urgent/action required list as described herein.

FIG. 7 is an image illustrating functionality of the scheduler for being able to enter a patient name, a patient date of birth (DOB), and/or a patient medical record number (MRN) to search for the patient.

FIG. 8 is an image illustrating that the schedule interface may require the scheduler to input one or more items of information associated with a new patient. For instance, the interface may present areas to enter a first name, a last name, a DOB, an MRN, an email address, a cell phone number, and/or combinations thereof for a new patient.

FIG. 9 is an image of the scheduler interface illustrating functions for determining if the patient has a qualifying ride.

FIG. 10 is an image of the scheduler interface for presenting questions to the patient.

FIG. 11 is an image of the scheduler interface illustrating a refusal by the patient to be scheduled.

FIG. 12 is an image of the scheduler interface of a list of encounters provided by the application to the scheduler.

FIG. 13 is an image of displaying through the application choices for procedures.

FIG. 14 is an image of displaying through the application choices for procedures.

FIG. 15 is an image of displaying through the application choices for procedures.

FIG. 16 is an image illustrating database storage of patient responses.

FIG. 17 is an image of the application showing an interface for providing patient responses to a plurality of questions associated with hospital history.

FIG. 18 is an image of the application showing an interface for providing patient responses to a plurality of questions associated with hospital history.

FIG. 19 is an image of the application showing an interface for providing patient responses to a plurality of questions associated with hospital history.

FIG. 20 is an illustration of the application depicting a drop-down menu for translating an event.

FIG. 21 is an image of the application illustrating querying the patient for detailed cardiac information.

FIG. 22 is an image of the application illustrating querying the patient for detailed cardiac information.

FIG. 23 is an image of the application illustrating querying the patient for detailed cardiac information.

FIG. 24 is an image of the application illustrating patient input of information related to diabetes and/or kidney complications.

FIG. 25 is an image of the application illustrating patient input of information related to pulmonary difficulties/events and other questions.

FIG. 26 is an example patient interface for answering the questions about any use of anticoagulants by the patient.

FIG. 27 is an example patient interface for answering the questions about any use of anticoagulants by the patient.

FIG. 28 is an image illustrating input of insurance information by the patient to the application.

FIG. 29 is an image of the application for procedure date input.

FIG. 30 is an image of the application for location input.

FIG. 31 is an image of an example embodiment interface where the scheduler has selected a location that is incompatible with previously entered information.

FIG. 32 is an image of the scheduler illustrating restrictions/requirements based on patient scheduling decisions.

FIG. 33 is an image illustrating the application presenting the user with a desired prep type for certain procedures.

FIG. 34A is an image of the “Maya Approval” flowchart described herein.

FIG. 34B is an image illustrating Maya Approval of the application as described herein.

FIG. 35 is an image of an example patient history interface associated with Maya approval.

FIG. 36 is an “Anticoagulant Approval” flowchart as described herein.

FIG. 37 is a “Cardiology Approval” flowchart as described herein.

FIG. 38 is a flowchart for “Additional Cardiology Evaluation” as described herein.

FIG. 39 is a flowchart for “Physician Approval.”

FIG. 40 is an “Insurance QA” flowchart.

FIG. 41 is an image of an example scheduler interface.

FIG. 42 is an image of an example scheduler interface.

FIG. 43 is an image illustrating that the application can notify the scheduler regarding a change in condition approvals and status.

FIG. 44 is an image depicting an exemplary change from a portion of the first set of instructions to the second set of instructions.

FIG. 45 is an illustration of a computing device which may be configured to execute functionality described herein.

FIG. 46 is an example of “Procedure Information”, which shows an example of the instructions that may be given.

FIGS. 47A-K are examples of “Procedure Instructions”, which guide the user through any preparation required for the procedure from “10 Days Prior”, “7 Days Prior”, “5 Days Prior”, “3 Days Prior”, “2 Days Prior”, and “1 Days Prior”.

FIGS. 48A-C are examples of “Day of Procedure”, which is part of the procedure instructions that may generate a “Day of Procedure” heading with corresponding instructions to the patient based on conditional inputs from the patient.

FIG. 49 is an example of a “Maya Approval Logic” check that the application applies.

FIGS. 50A-B consist of an example of a “Anticoagulant Logic” check that the application applies.

FIGS. 51A-B are examples of a “Prep Type Logic” that the application applies in association with the prep type.

FIGS. 52A-D are examples of dynamic instructions for the patient to follow based on the stored data structures.

Corresponding reference characters indicate corresponding elements among the view of the drawings. The headings used in the figures do not limit the scope of the claims.

### DETAILED DESCRIPTION

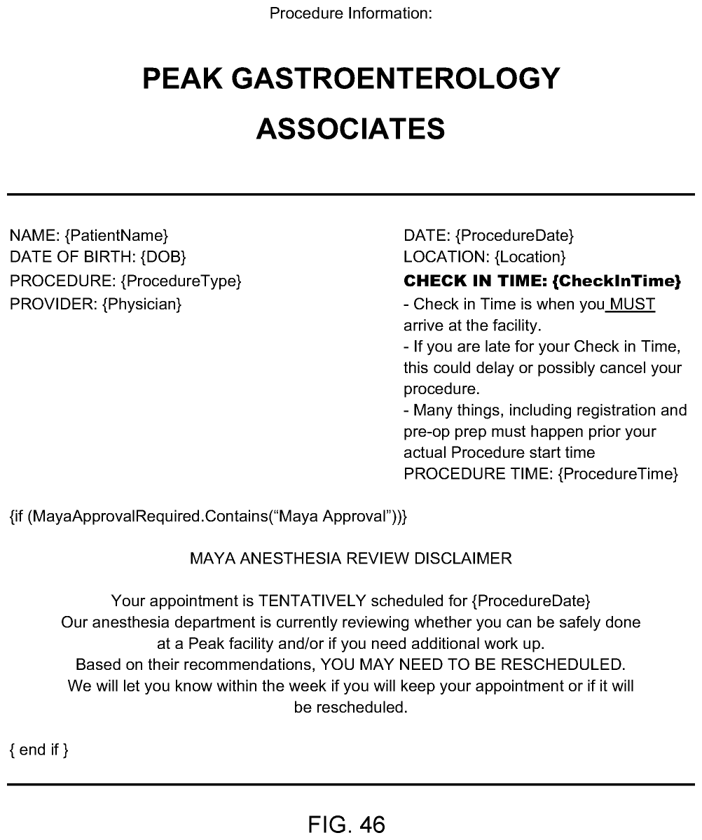

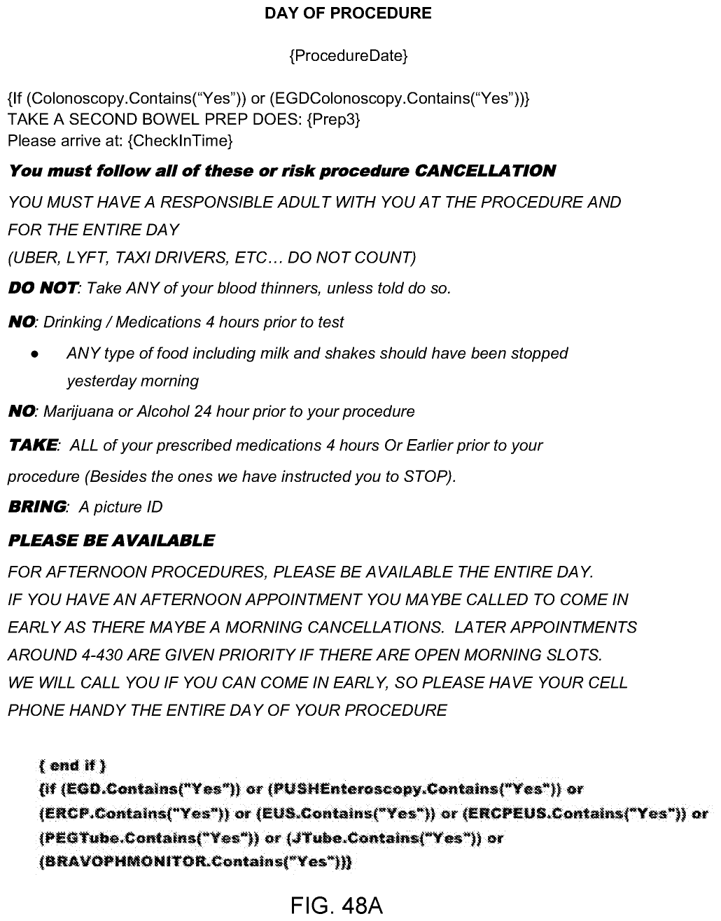

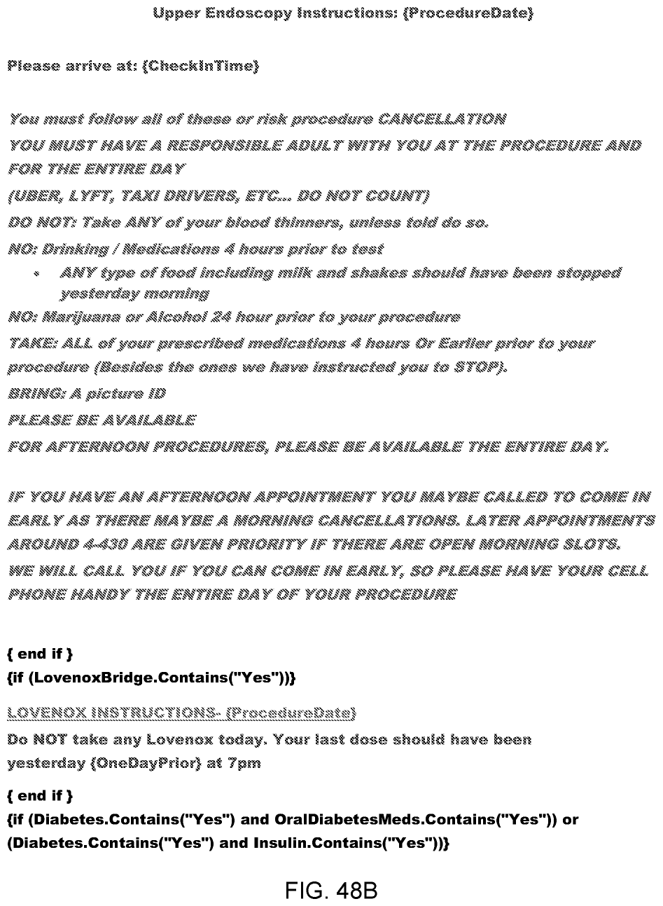

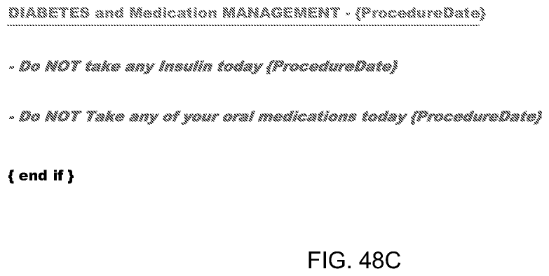

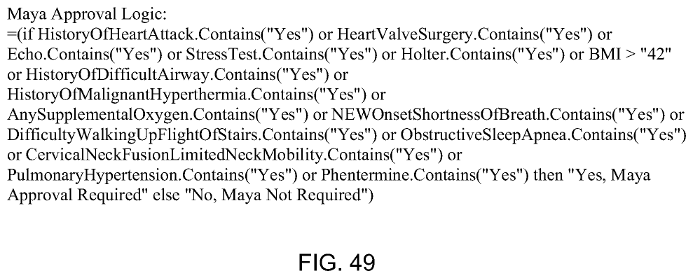

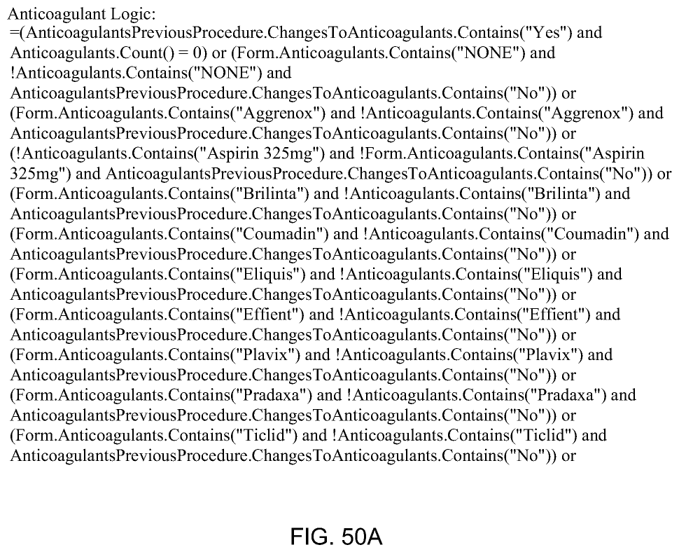

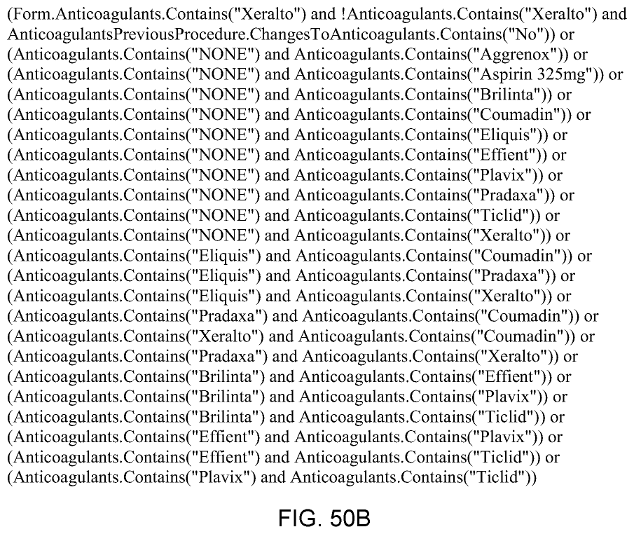

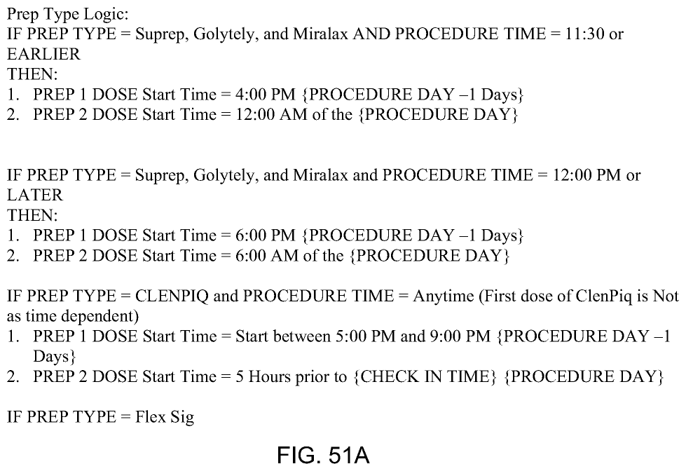

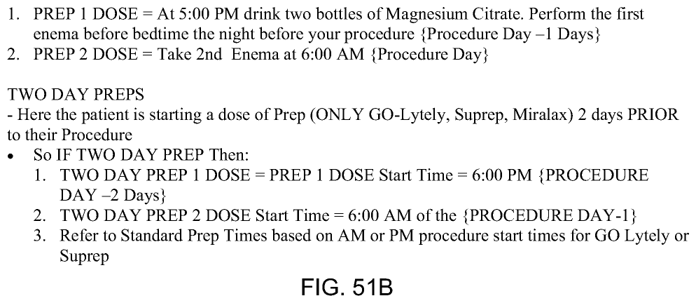

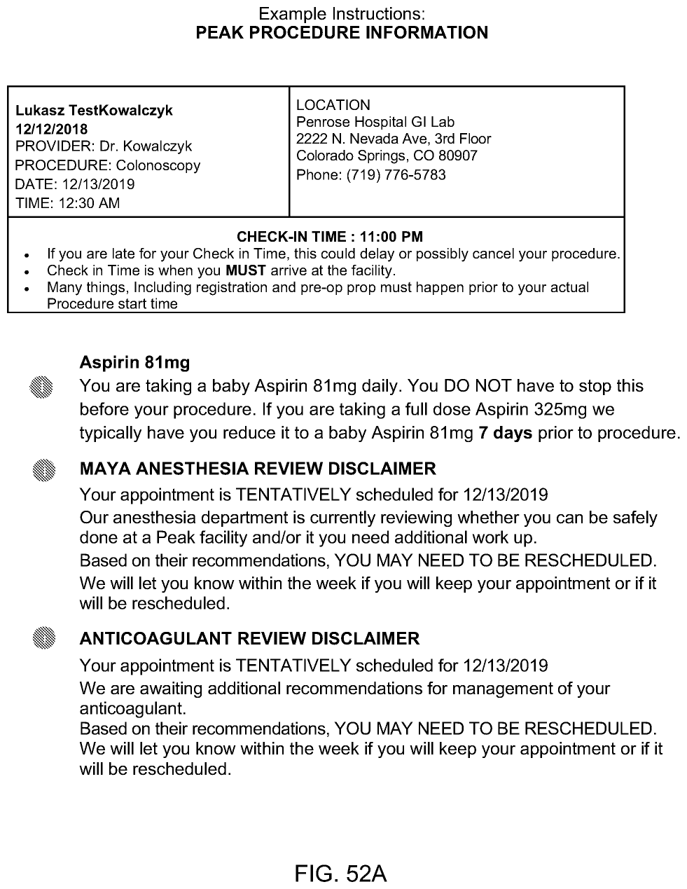

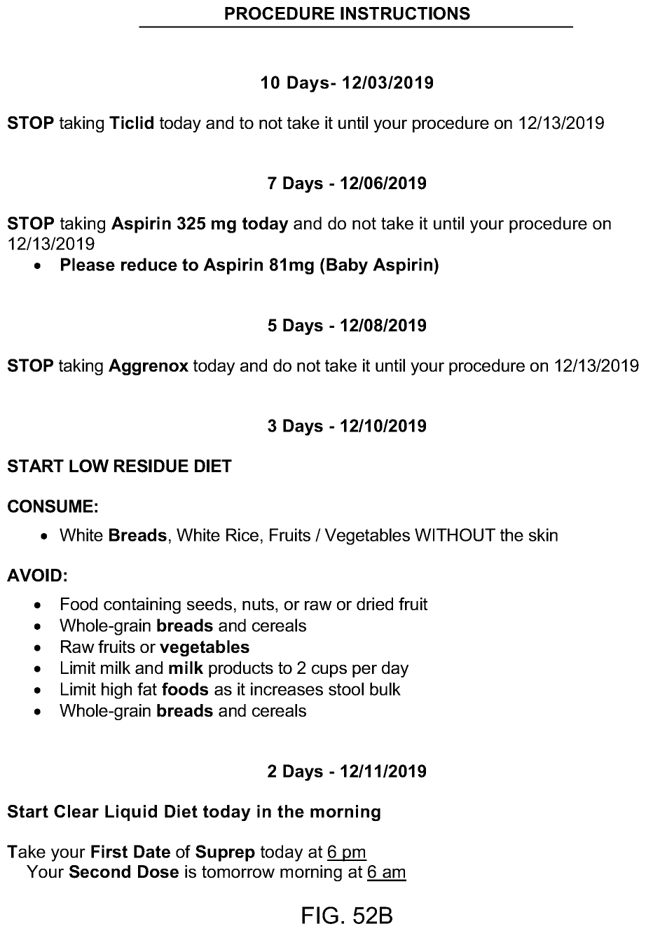

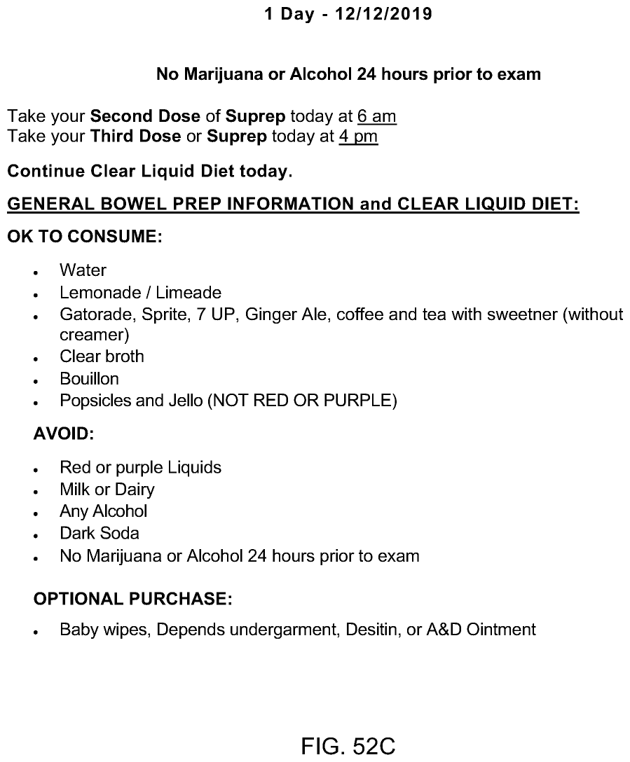

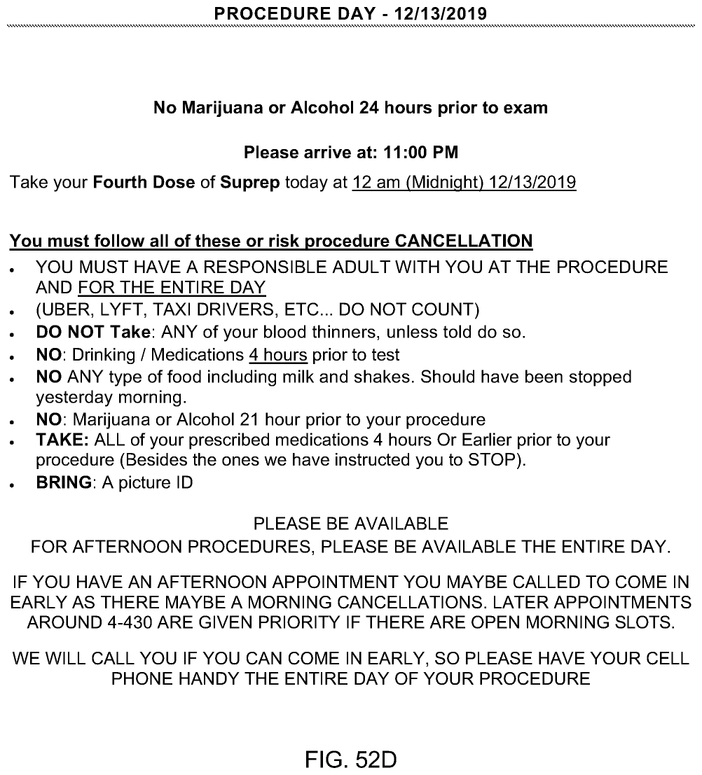

Embodiments of the present disclosure reference the Drawings including Exemplary Logic and Supplemental information of FIGS. 46-52D provided therein, and generally relate to a dynamic application with adaptable instructions. Before any embodiments of the disclosure are explained in detail, it is to be understood that the disclosure is not limited in its application to the details of construction and the arrangement of components set forth in the following description or illustrated in the corresponding flowcharts. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

The term “encounter” may describe an event or series of events that occur on a specific date at a specific time. An encounter may be the following: a patient visiting either a clinical facility or a hospital, a patient checking in, and a patient undergoing general anesthesia. Each event or series of events may be represented as a single encounter in a medical provider system. Each encounter may have a set of instructions associated therewith. Encounters may be subject to change or rescheduling, as well as cancellation. Any follow-up procedures may be classified as different encounters in the medical provider system. Each encounter may be facilitated by one physician.

The term “procedure” may include one or more medical procedures performed within one encounter. This may include procedures in addition to the primary procedure.

The term “scheduler” may refer to a person who creates records of encounters in the medical provider system.

The term “physician” may refer to the medical professional who has access to a read-only version of a patient's encounter history.

As used herein, any reference to any term or phrase containing the word “Maya” should be considered equivalent to a reference to anesthesia and/or an anesthesiologist and should carry the same meaning. For instance, the term “Maya Approval” may denote the approval of an anesthesiologist.

The term “Maya approver” may refer to a person who has access and authority to conduct Maya Approval.

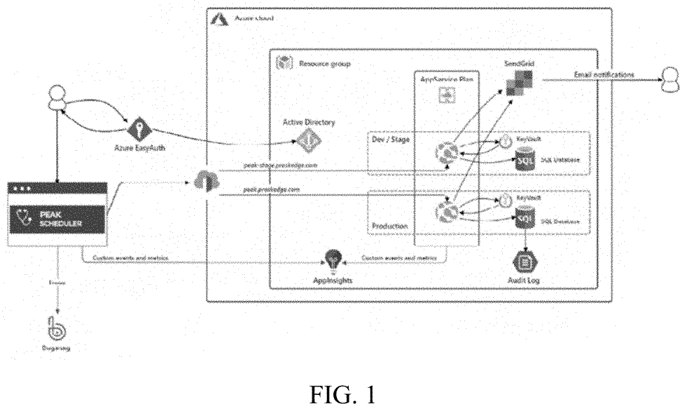

The present disclosure includes a system that may provide a set of dynamic instructions to a patient. One embodiment of the system is shown in FIG. 1. As noted above, the system may comprise a scheduler and an application. The application may comprise a scheduler interface and a cloud network. The scheduler, as previously defined, may be a person responsible for creating and documenting one or more encounters. The scheduler interface may allow the scheduler to create an encounter. The cloud network may permit an application to communicate information. For instance, the application may be configured to utilize the cloud network to access a database associated with patient information. While the embodiments of the present disclosure may be described with reference to a database, a single database should not be viewed as limiting, and multiple databases may be present. The database may contain multiple data structures associated therewith. In one embodiment, the database may be a Structured Query Language (SQL) database, and may be accessed by one or more keys to retrieve information therein. In some embodiments, the application may access the patient information in generating the dynamic instructions. In one embodiment, the application may query the patient for answers to a range of questions, and store the answers in the database. In some embodiments, the application may query the patient for a relevant medical history and/or access the database to obtain the relevant medical history before permitting the scheduler to schedule an encounter.

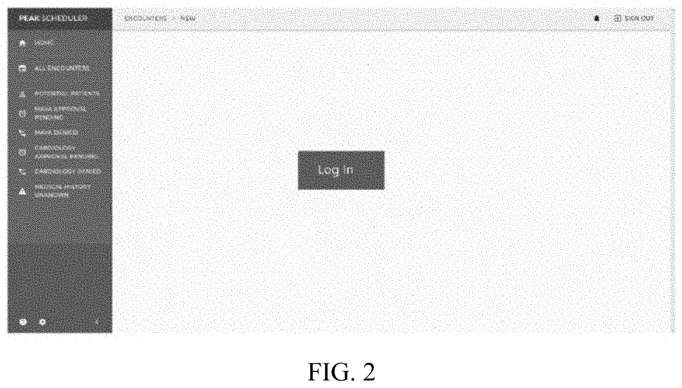

The scheduler interface may provide a scheduler with a login screen. As shown in FIG. 2, the login screen may comprise a login button, which, when selected by the scheduler, may prompt the entering of a username and a password in order to access the scheduler interface.

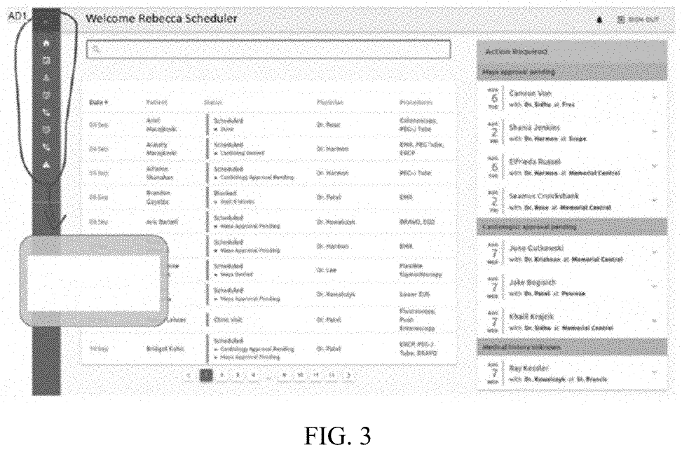

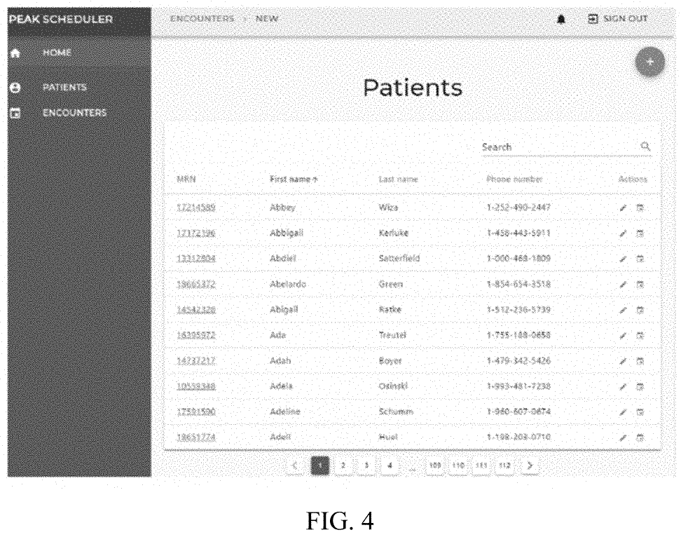

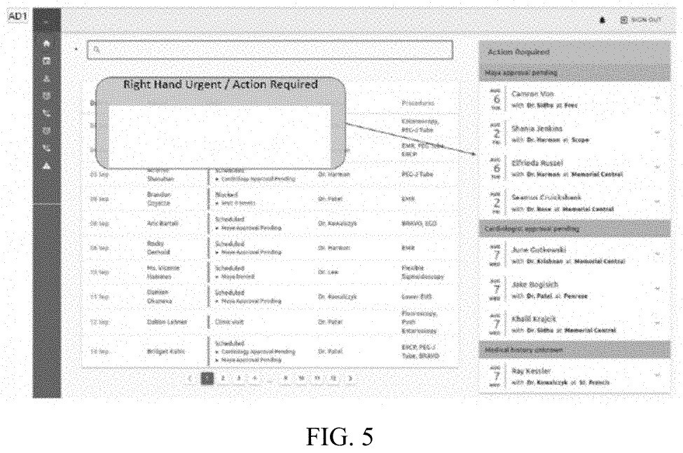

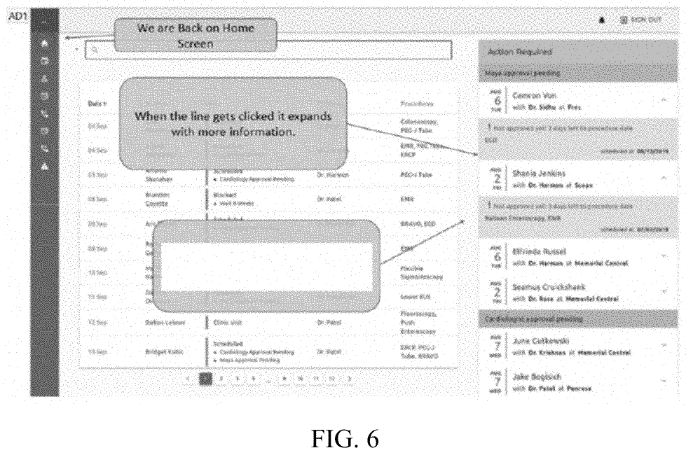

Upon correct entry of the username and the password, the scheduler interface may transition the scheduler to a home screen. As illustrated in FIGS. 3-4, the home screen may comprise a patients list, a create new patient button, an encounters list, and a do to list, and an urgent/action required list. Two example home screens are shown in FIGS. 3-4.

The to do list and the urgent/action required list may be viewed in the images below. The scheduler may be able to access additional information by selecting any of the encounters on the urgent/action required list.

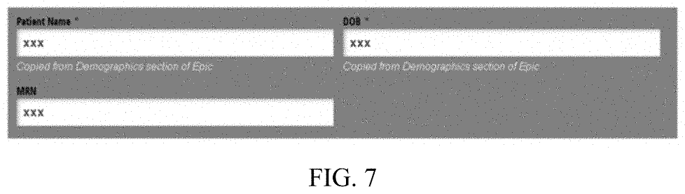

The scheduler may be able to add use the schedule interface to create a new encounter for an existing patient. For instance, as shown in the images of FIGS. 3-4, the scheduler may be able to search for existing patients. In some embodiments, the scheduler may be able to enter a patient name, a patient date of birth (DOB), and/or a patient medical record number (MRN) to search for the patient, as indicated in FIG. 7. The search may cause the schedule interface to query the application, which may then access the database, through the use of a processor, to locate any patient that contains matching information with the search data entered by the scheduler. The application may then return the patient name and any encounters scheduled therefor.

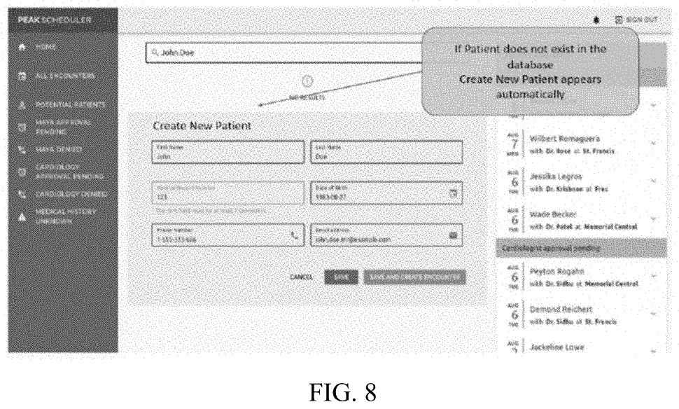

The schedule interface may permit the scheduler to create a new patient by selecting a create new patient button. Upon selecting the create new patient button, the schedule interface may require the scheduler to input one or more items of information associated with a new patient. For instance, the interface may present areas to enter a first name, a last name, a DOB, an MRN, an email address, a cell phone number, and/or combinations thereof for a new patient, as shown in FIG. 8. In some embodiments, the application may automatically begin the addition of a new patient when the scheduler searching for a patient whose information have not been previously stored in the database.

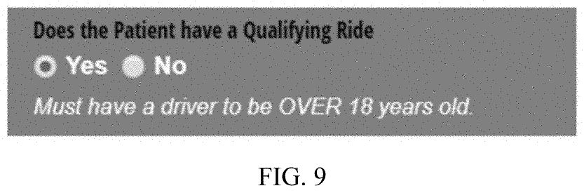

The schedule interface may provide a patient list and a start new encounter button next to each patient. Upon selecting the start new encounter button, the application may prompt the scheduler to answer one or more questions to create the new encounter. For instance, a first question may be “does the patient have a qualifying ride?” as shown in FIG. 9. The first question may indicate whether the patient would have a ride home from the facility following the procedure. If the answer is “No”, the application may cancel the scheduling of the new encounter. This may be because the patient may be incapable of operating a vehicle after the encounter, and may need someone else to drive them home.

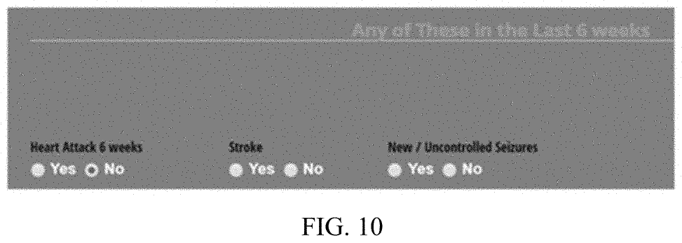

If the answer is “Yes”, the application may then present the scheduler medical events questions regarding recent medical events, through the scheduler interface. For instance, the application may present the question “Any of these in the last 6 weeks?”, as noted in FIG. 10. The scheduler may be then able to answer “Yes” or “No” to the medical events questions.

As the image in FIG. 10 details, the application may query about any recent heart attacks, strokes, and/or any new or uncontrolled seizures. If the scheduler selects “Yes” to any of the questions, the application may cancel the scheduling of the new encounter.

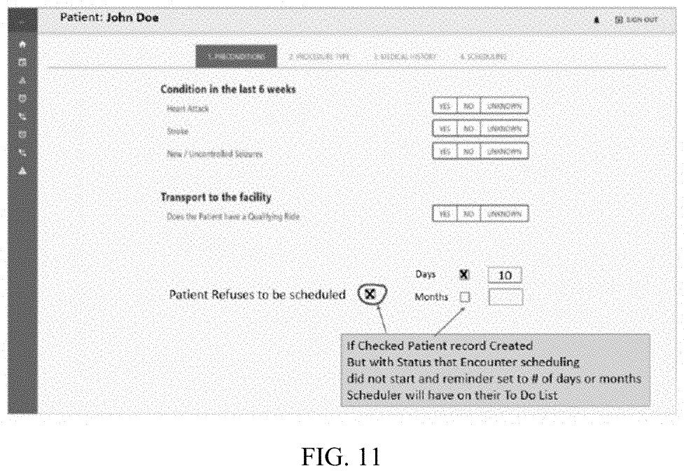

Additionally or alternatively, as noted in the image of FIG. 11, the patient may refuse to be scheduled for the encounter. Accordingly, the scheduler may be provided an option for indicating to the application that the patient has refused the scheduling, and may optionally enter a reminder to attempt another scheduling after the passing of a certain time period. In the exemplary embodiment noted in the above, the scheduler has set a reminder for 10 days, after which the scheduling may appear in the to do list for the scheduler.

Once the scheduler or patient has answered the first question and the medical events questions, the application may generate an encounter. It may apply a “follow up” tag to the encounter. In some embodiments, the application may generate the encounter with a “follow up” tag regardless of the responses to questions about the ride home and the recent medial events.

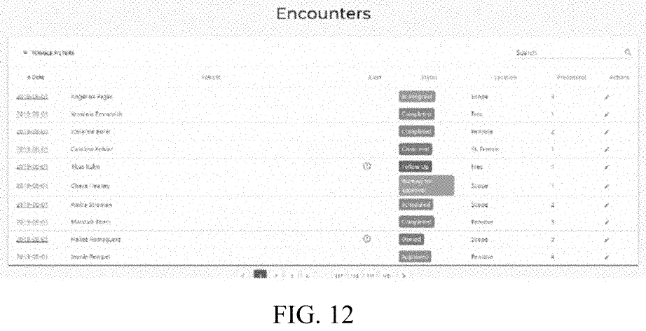

As noted in FIG. 12, the application may provide, through the schedule interface, a list of encounters to the scheduler. The list of encounters may contain a plurality of columns containing information regarding the encounter, while each row in the list may be a separate encounter. The schedule interface may provide a date of the encounter, a patient name, a location of the encounter, the number of procedures to be completed in the encounter, an alert indicator, and an actions column. The actions column may allow the scheduler to alter the details associated with the encounter, such as the date and location of the procedure.

Each encounter may have a status associated therewith. The status may have a label to inform the scheduler of the current status of the encounter. The label may be one of a plurality of possible labels. For instance, an “In Progress” label may be present on the encounter, indicating that a record of the encounter has been created but has not been completed. The “In Progress” label may indicate that not all the required patient information has been filled in, and that all fields may be edited to complete the scheduling of the encounter.

A “No Ride” label may indicate that the patient does not have a qualified ride.

A “6 Weeks” label may indicate that one or more of the answers to the “Any of these in the last 6 weeks?” question may have been “Yes”.

A “Scheduled—Done” label may indicate that all patient fields have been completed, a date and a time for the encounter have been selected, and no additional approvals or data are needed to proceed.

A “Scheduled—Maya Approval Pending” label may be used when the all information is entered, but Maya Approval is required to proceed.

A “Scheduled—Done, Maya Approved” may be used when all information is entered, and Maya Approval has been granted.

A “Scheduled—Medical History Unknown” may be used when all information has been entered except one or more components of the medical history of the patient.

A “In Progress—Maya Denied” label may be associated with an encounter that Maya has denied. In this instance, the application may then display the encounter in a “To Do” list for the scheduler. In some embodiments, the scheduler may attempt to schedule the encounter at a different hospital, or may cancel the encounter.

A “Cancelled” label may be used when an encounter is cancelled. In some embodiments, the application may provide a textbox to allow the scheduler to enter text describing the reason for the cancellation.

A “Completed Success” label may be used for a successful encounter where all procedures in the encounter were completed without any complications occurring.

A “Completed with Problems” label may be utilized when the procedures were completed, but complications may have occurred. In some embodiments, the application may provide a textbox to permit the scheduler to enter text describing the complications.

A “Clinic Visit Needed” label may be associated with an encounter that has no date and/or time because a clinic visit is required before an encounter can be scheduled. During the clinic visit, a physician may decide the next steps required and communicate this information to the scheduler.

A “Scheduled—Cardiology Approval Pending” label may be utilized when an encounter can be scheduled, including a date and time, but a separate process may validate with a cardiologist that any patient anticoagulant medications may be stopped.

The labels indicated above are not particularly limiting, and alternative or additional labels may be utilized by embodiments of the present disclosure.

In some embodiments, one or more of the encounters may be edited. In one embodiment, all encounters except for encounters with the “Cancelled” label or the “Completed Success” label may be edited. The application may contain conditional checks to ensure that any encounter not containing the “Cancelled” label or the “Completed Success” label is still updatable and that any instructions that issue therefrom are still dynamically updatable. In some embodiments, the application may implement a similar logic when editing an encounter that it does when creating an encounter.

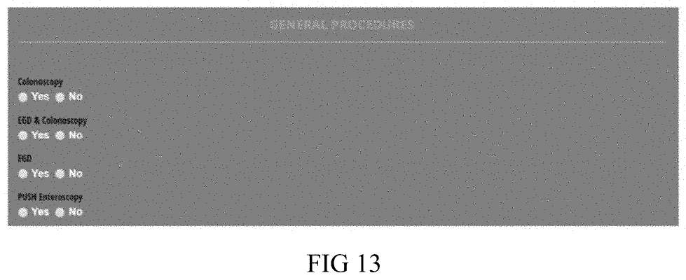

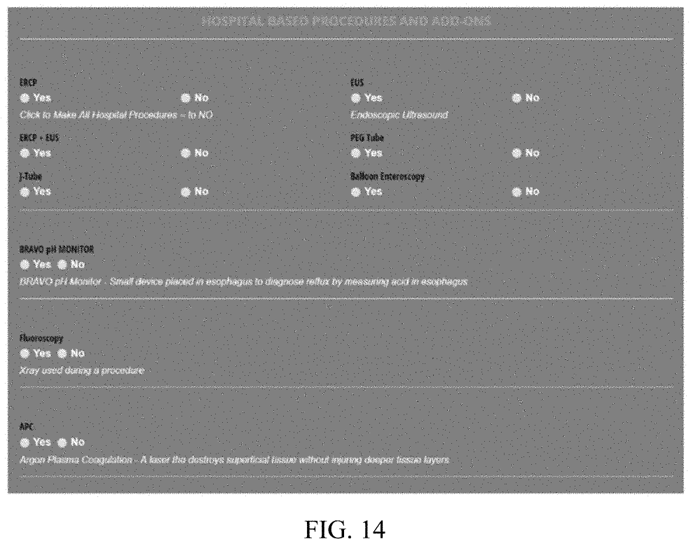

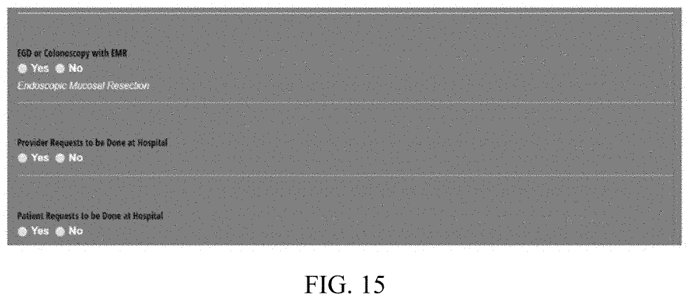

The application may allow a patient to select which types of procedures may be completed during an encounter (e.g., during a single visit to the hospital). As previously noted, the application may present the questions to the patient before permitting the scheduling of the encounter. The application may display to the patient the choices for procedures shown in the images of FIGS. 13-15.

As seen in the images of FIGS. 13-15, the patient may be able to elect one or more general procedures. For instance, the patient may choose either “Yes” or “No” to the following procedures and/or combinations thereof: a colonoscopy, a combined esophagogastroduodenoscopy (EGD) and colonoscopy, an EGD, and/or a push enteroscopy.

Moreover, the patient may elect one or more hospital-based procedures and add-ons. As noted above, the patient may be able to select either “Yes” or “No” to the following procedures and add-ons and/or combinations thereof: an endoscopic retrograde cholangiopancreatography (ERCP), an endoscopic ultrasound (EUS), a combined ERCP and EUS, a percutaneous endoscopic gastrostomy (PEG) tube, a jejunostomy tube (J-tube), and/or a balloon enteroscopy. The application may additionally or alternatively provide the patient with a button to set all the procedures and add-ons to the “No” state.

The application may additionally or alternative prompt the patient to answer “Yes” or “No” to the following add-ons and/or combinations thereof: a pH monitor, fluoroscopy, Argon Plasma Coagulation (APC), an EGD or colonoscopy with Endoscopic Mucosal Resection (EMR).

The application may be equipped to determine invalid combinations of procedures. For instance, if the patient were to select an invalid combination, the application may first check to ensure the combination of procedures is invalid, and may then display this information to the patient. For instance, the application may display, through the user interface, a message such as “You are scheduling procedures that are not typically scheduled together. Please confirm the reason for scheduling the procedures during the same encounter.” The application may then provide the patient with a textbox to enter text related to the scheduling reason. The application may then update the label associated with the encounter to the “Clinic Visit Needed” label, to ensure a medical professional approves of the combination of procedures.

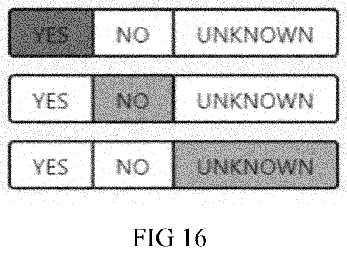

The application may store the patient responses in the database. For instance, the application may store a binary response for each question (i.e., for each question, the application stores in the database a 1 for a “Yes” and a 0 for a “No”). As noted in the image of FIG. 16, all records in the medical history of the patient may have 3 statuses: Yes, No, and Unknown. The Unknown field may be a temporary value, and may indicate that the field is not filled in when determining which label to assign to the encounter. The Unknown field may assign a 0 for the purpose of determining conditional operations in the application workflow.

In some embodiments, the application may provide the patient with a method of entering detailed information related to their medical history. For instance, the application may provide the patient with a textbox to enter information, such as a date or year of a heart attack. Upon the user entering text into the textbox, the application may save the text to the database. The text may later be displayed to the Maya Approver and/or the physician to provide more detailed information to assist the Maya Approver and/or the physician in exercising medical judgment for the encounter.

In some embodiments, the application may prevent the scheduler from viewing the exact patient data, and may only label the encounter for the user to view based on the patient responses. In some embodiments, the application may logically determine a required label before providing the label to the scheduler through the scheduler interface.

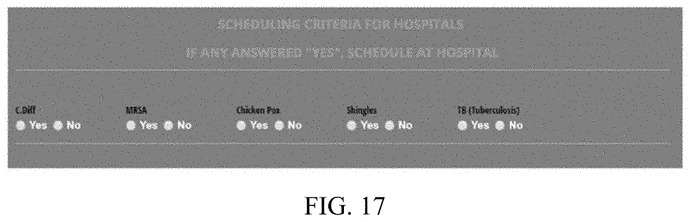

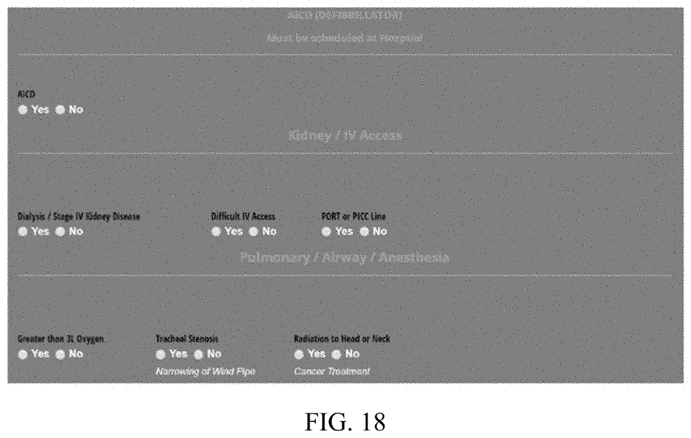

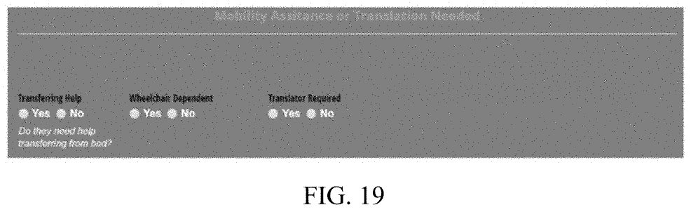

The application may further provide the patient with an interface to enter hospital history associated with the patient. The interface may comprise a plurality of questions, as shown in the images of FIGS. 17-19.

As seen in FIGS. 17-19, the application may prompt the patient to disclose information related clostridioides difficile (C. diff), methicillin-resistant Staphylococcus aureus (MRSA), chicken pox, shingles, and/or tuberculosis (TB). If the patient has or had any of the above-mentioned conditions, the application may require the patient procedures be performed at the hospital. When the scheduler begins the scheduling of an encounter for the patient, the application may, through a processor, access the database. In the event that any 1 entry appears in the database (i.e., the patient answered “Yes” to any of the above-mentioned questions), the application may dynamically update the encounter to indicate that the procedure should be performed at the hospital.

Moreover, the patient may be required to answer “Yes” or “No” to whether they have an automated implantable cardioverter defibrillator (AICD). If the answer is “Yes”, the application may require that the encounter be scheduled at the hospital.

The application may also prompt the patient to answer questions about any dialysis requirements or kidney disease, any complications that may arise from difficult intravenous (IV) access, or any port or peripherally inserted central catheter (PICC) line for IV access.

The application may also require the patient to answer questions regarding any pulmonary or airway requirements. For instance, the application may ask the patient to enter “Yes” or “No” to whether the patient requires more than 3 liters (L) of oxygen, if the patient has tracheal stenosis, and if the patient has had any procedure requiring radiation to the head or neck.

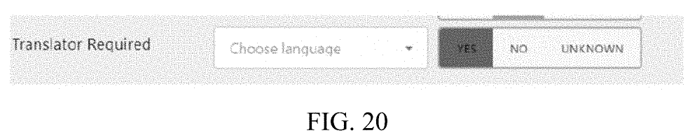

The application may also inquire about any additional limitations or amenities the patient may require. These may include assistance when moving to and from a bed, wheelchair dependence, and/or translation services. In the event a translator is required, the application may provide the patient with a drop-down menu to select a desired language, as shown in the illustration of FIG. 20.

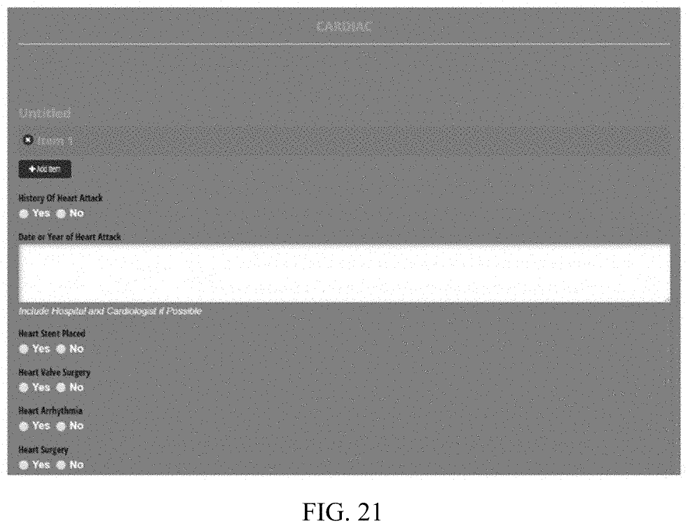

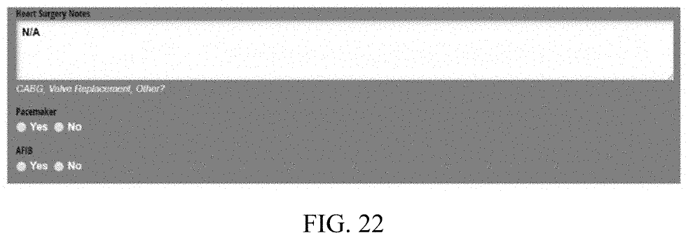

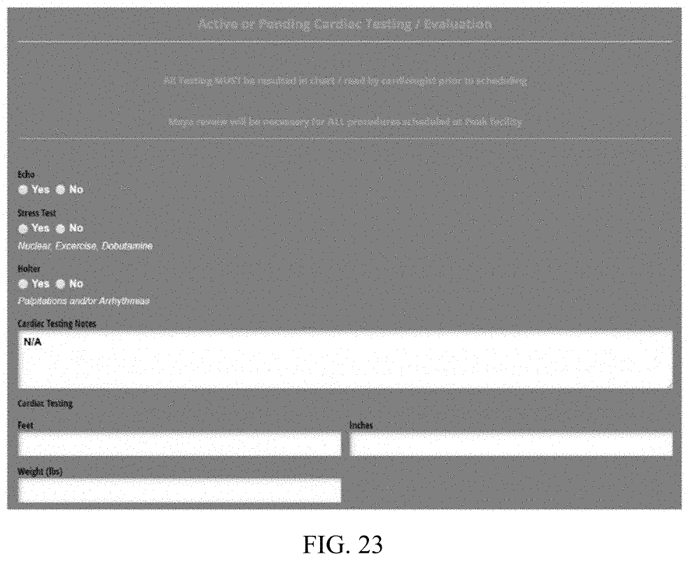

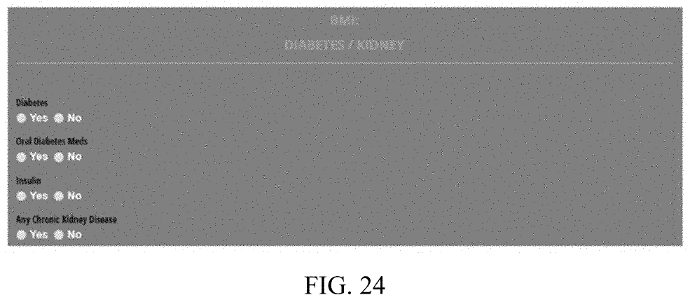

The application may also query the patient for detailed cardiac information. For example, the application may provide the patient with an interface to enter patient information related to cardiac events, as shown in the images of FIGS. 21-23.

As illustrated, the application may request that the patient enter information related to any history of heart attacks experienced by the patient. If the patient answers “Yes” to a history of heart attack question, the application may request more information about the heart attack. For instance, the application may provide the user with a textbox to enter the date or year of the heart attack, as well as the hospital and/or cardiologist associated with the incident. The application may also prompt the patient to answer questions related to whether the patient has a heart stent, has undergone heart valve surgery, has heart arrhythmia, has had any heart surgery, has a pacemaker, and has atrial fibrillation (AFIB). The patient may be provided with a textbox to add any notes related to any heart surgery.

The application may further permit the patient to enter information related to active or pending cardiac testing or evaluation. For instance, the patient may be able to enter information regarding whether the patient has recently been subject to an echo, a stress test, and/or a Holter monitor. The patient may have the option to provide testing notes related to one or more of the cardiac tests, as well as patient information, such as patient height and/or patient weight.

As seen in the image of FIG. 24, the patient may be able to further enter information related to diabetes and/or kidney complications. For instance, the patient may be able to answer “Yes” or “No” as to whether the patient has diabetes and, if yes, whether the patient uses an oral medication therefor, uses insulin treatment, and has any chronic kidney disease.

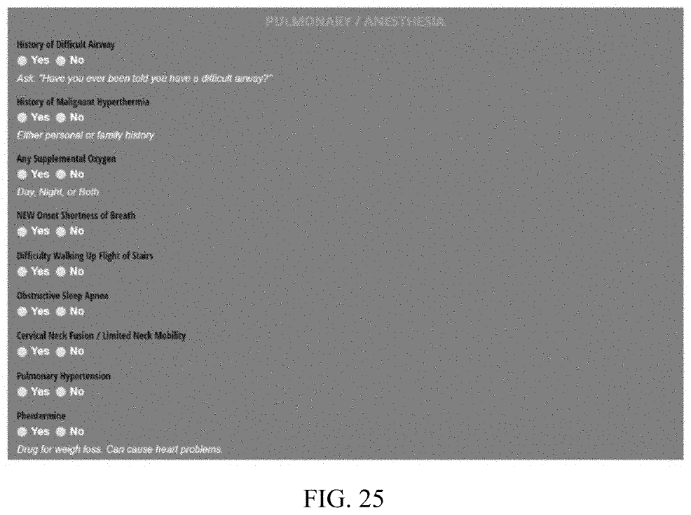

Additionally or alternatively, the application may request patient information related to pulmonary difficulties/events and other questions that may be beneficial to the anesthesiologist. As shown in FIG. 25, the application may request patient responses to one or more questions.

The application may prompt the patient to enter information related to a difficult airway; the application may ask something to the effect of “Have you ever been told you have a difficult airway?” The patient may also be asked to enter information related to history of malignant hyperthermia. This question may pertain not only to the patient, but also to any family history thereof.

The application may request the patient answer a question related to oxygen use. The oxygen use question may pertain to whether the individual uses supplemental oxygen during the day, during the night, or both. The application may question the patient about any or all of the following: new onset shortness of breath, difficulty walking up a flight of stairs, obstructive sleep apnea, cervical neck fusion and/or limited neck mobility, pulmonary hypertension, and phentermine use.

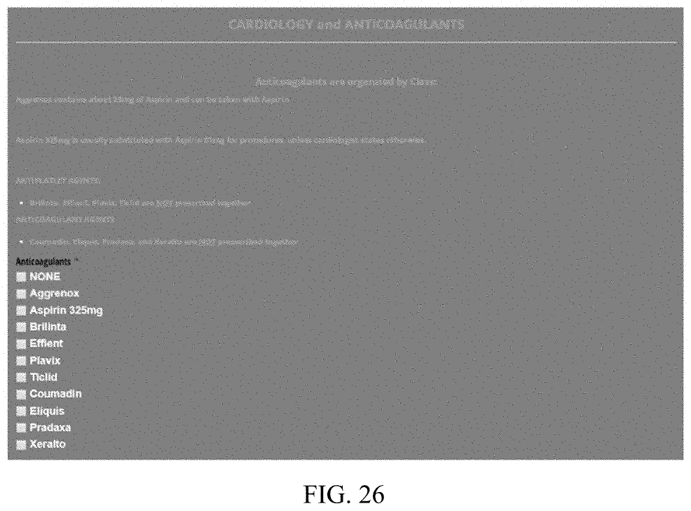

The application may further query about any use of anticoagulants by the patient. An example patient interface for answering the questions is shown in FIG. 26.

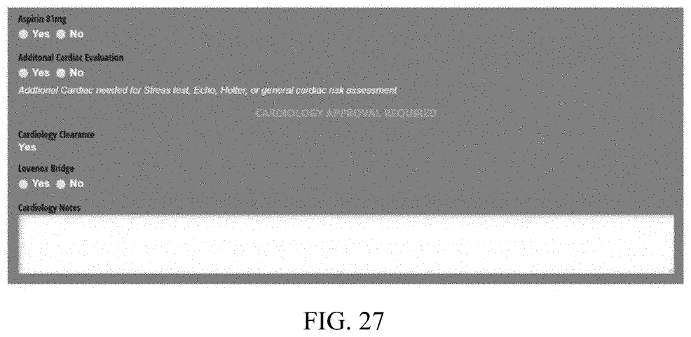

As noted in the FIGS. 26-27, the application may prompt the patient to select any and all anticoagulants currently being taken by the patient. For instance, the patient may be able to select one or more anticoagulants to reflect the current anticoagulants taken by the patient. The list of anticoagulants may be organized by class. For instance, the list may present first as set of antiplatelet agents, such as Ticagrelor (brand name Brilinta), Prasugrel (brand name Effient), Clopidogrel (brand name Plavix), and Ticlopidine (brand name Ticlid); the list may then continue to anticoagulant agents such as Warfarin (brand name Coumadin), Apixaban (brand name Eliquis), Dabigatran (brand name Pradaxa), Rivaroxaban (brand name Xarelto). The list may also contain other medications such as Aspirin in various concentrations, as well as additional medications such as a combination of Aspirin and Dipyridamole (brand name Aggrenox). It should be noted that the medications listed here are in no way limiting, and additional or alternative medications may appear to the patient. Furthermore, the order of the medications listed is not to be interpreted as limited to the embodiment noted above, and the medications may be presented in any order.

The application may provide the patient with information related for anticoagulants. For instance, the application may indicate to the patient that Brilinta, Effient, Plavix, and Ticlid are not prescribed together. Similarly, the application may inform the patient that Coumadin, Eliquis, Pradaxa, and Xeralto are not prescribed together. Accordingly, if the user selects conflicting medications, the application may notify the user that an ineligible combination has been selected, and may prompt the user to adjust the anticoagulant choices to change or remove the ineligible combination. The application may display the ineligible combination to the patient through the user interface, and prompt the patient to correct the ineligibility.

The application may prompt the user to enter information regarding Aspirin 81 mg. Aspirin 81 mg may be the concentration of Aspirin prescribed to the patient to help prevent blood clots. Aspirin 81 mg is typically not stopped before procedures, unless the patient is currently taking another antiplatelet agent, in which case the Aspirin 81 mg is typically stopped. Stopping the Aspirin 81 mg typically requires clearance from a cardiologist.

The application may additionally require information about any additional cardiac evaluations the patient may need. The additional cardiac evaluations may be, but are in no way limited to, the echo test, the stress test, and/or the Holter monitor. Additionally or alternatively, the additional cardiac evaluations may be associated with a general cardiac risk assessment.

The application may ask about the use of using Lovenox as a bridge for the encounter. Lovenox is a short-term anticoagulant that is often used to minimize the time the patient is off their blood thinner. Lovenox is typically given the night before the procedure, but not on the day of the procedure. In some embodiments, the use of the Lovenox bridge may be conditional upon cardiology approval.

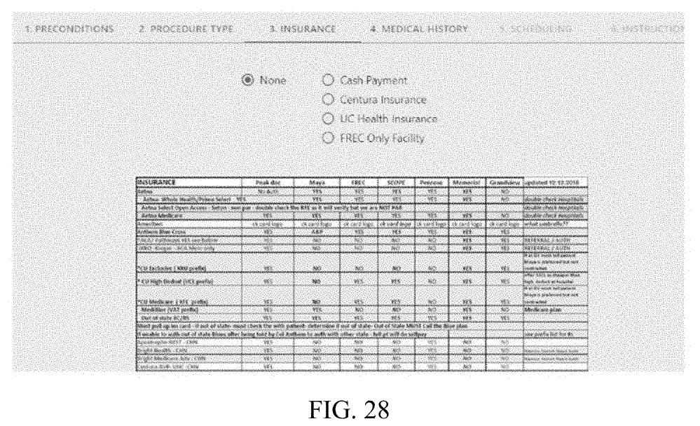

The application may require the patient to input insurance information. In some embodiments, the application may display a Portable Document Format (pdf) of insurances accepted by various facilities, as noted in the image of FIG. 28.

The application may permit the patient to enter their insurance provider. In one embodiment, the application may prompt the patient to answer what type of payment or insurance they will use for the procedure.

The choice of insurance may limit which locations the patient can choose to undergo the encounter. In one embodiment, the choice of Centura insurance may limit the choice of location to only Penrose or St. Francis locations. The application may automatically, upon the choice of Centura insurance, enter a 0 into the data structure in the database associated with all other locations other than the Penrose and St. Francis locations, effectively preventing the user from selecting a location other than Penrose or St. Francis. Additionally or alternatively, the choice of UCH insurance may only permit the Memorial location from being selected. The application may automatically, upon the selection of UCH, enter a 0 into the data structure in the database associated with all other locations other than the Memorial location.

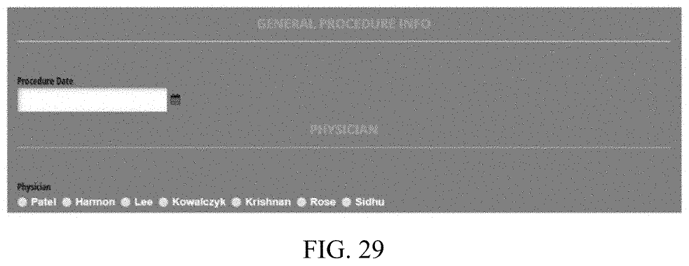

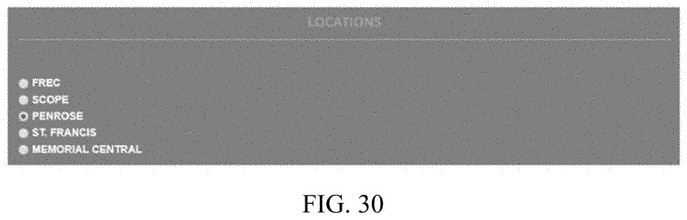

Referring to FIGS. 29-30, the application may request a procedure date input. For instance, the application may provide the scheduler with a calendar, and permit the scheduler to select a date for the encounter. In some embodiments, the scheduler may be able to directly input a date through the use of a textbox. The scheduler may also be able to select the physician who will be performing the one or more procedures.

The user may be able to enter a location for the encounter. For instance, the application may present the scheduler with a plurality of options for locations. The options may be conditionally based on the patient's previous inputs for choice of insurance, patient history, procedure type, and/or entered preferences, or combinations thereof. In some embodiments, Maya Approval may determine which locations are valid for the procedure.

In one embodiment, there may be 5 locations at which the patient may be scheduled. The locations may be divided into two categories: peak facilities and hospitals. FREC and SCOPE may be peak facilities, which may operate as outpatient Ambulatory Surgical Centers (ASC). The peak facilities may be designed for patients with minimal health issues, and for simple procedures. Penrose, St. Francis, and Memorial Central may be hospitals, and may be reserved for more medically complex patients.

The application may also prompt the patient to answer “Yes” or “No” to whether the patient requests that the procedures be done at the hospital. The application may also prompt the scheduler to answer “Yes” or “No” when scheduling the encounter as to whether the provider requests that the procedures be done at a hospital. The responses to the above questions may prevent the scheduler from selecting one or more locations for the encounter. For instance, if the patient requests that the procedures be done at the hospital, the application may automatically prevent the scheduler from scheduling the encounter at a location that is not the hospital by way of entering a 0 into a data structure in the database associated with locations that are not hospitals.

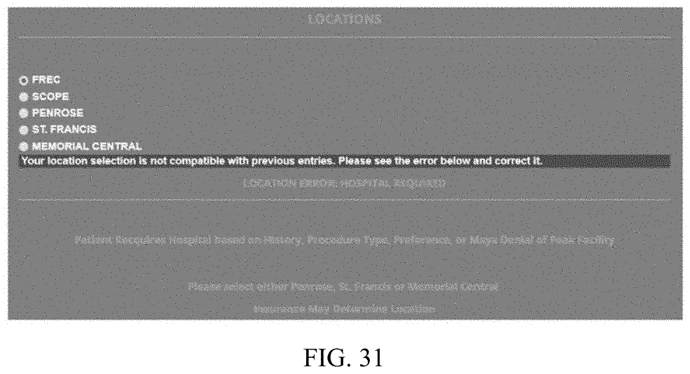

The image in FIG. 31 is an example embodiment interface where the scheduler has selected a location that is incompatible with previously entered information. As noted above, the selection of the FREC location by the user has resulted in an error. The application may alert the user to the error through the use of a pop-up text. The pop-up text may indicate the error, and provide the user with guidance to correct the error. In the image of FIG. 31, the location may be incompatible because the user selected an insurance provider that is not compatible with the currently selected location. In some embodiments, the application may omit displaying locations that the application has conditionally determined to be incompatible with the patient information stored in the database. For instance, the application may access the database and, through the processor, determine that the one or more of the patient responses related to the choice of insurance, patient history, procedure type, and/or entered preferences are incompatible with one or more of the locations. The application may then omit displaying the one or more locations to the scheduler when the scheduler attempts to schedule the encounter.

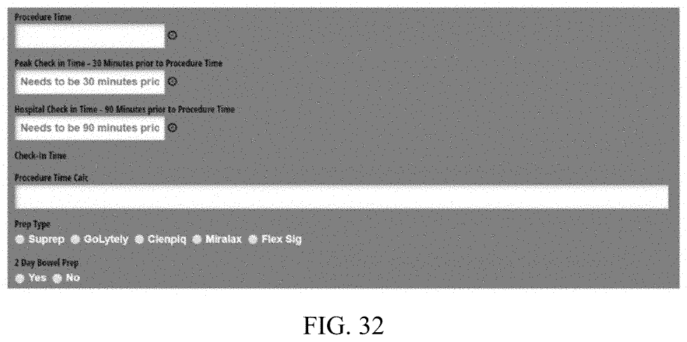

Referring to FIG. 32, once the location has been selected, the scheduler may enter a procedure time. The procedure time may be the time at which the patient will undergo the one or more procedures in the encounter. The scheduler may be able to enter the procedure time by manually inputting the time through an input device. In some embodiments, the application may provide the scheduler with a drop-down menu to select a desired procedure time.

The application may permit the scheduler to determine a check-in time relative to the procedure time. In some embodiments, the scheduler may have to enter a check-in time a pre-determined amount of time prior to the procedure time. The check-in time may be conditional as to the location of the procedure. For example, the scheduler may require a check-in time 30 minutes prior to the procedure for the Peak facilities. However, if the procedure is to occur at any of the hospital locations, the check-in time may be 90 minutes prior to the procedure. The application may provide a drop-down menu to permit the scheduler to enter the check-in time. In some embodiments, the application may determine the check-in time conditioned on at least the location of the procedure by accessing the data structures in the database associated with the location of the encounter. In some embodiments, the application may automatically enter the check-in time for the scheduler based on the procedure time.

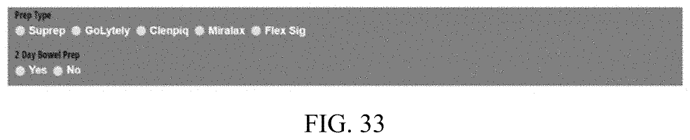

Referring to FIG. 33, in some embodiments, the application may present the user with a desired prep type for certain procedures (e.g., a colonoscopy). The user may elect the prep type the user plans to implement in preparation for the procedure, as well as whether the user will implement a two day bowel prep. For example, the application may present the user with a choice among Suprep, GoLytely, Clenpiq, Miralax, or Flex Sig. The choices are not limited to the embodiment mentioned above, and alternative and/or additional choices are possible. The choices elected by the user may be utilized conditionally when the application generates and/or updates the set of instructions to the patient. The user may also elect whether or not to engage in a 2-day bowel prep. In some embodiments, only GoLytely, Suprep, and Miralax provide the 2 day bowel prep, and the application may not display the 2 day bowel prep question to the patient if the patient has not selected GoLytely, Suprep, or Miralax.

The application may apply a logic associated with the prep type. The logic associated with the prep type may be described with reference to the “Prep Type Logic” described below in the Drawings.

The application may conditionally determine if the patient elected Suprep, GoLytely, or Miralax, and the procedure time is a morning session (i.e., 11:30 A.M. or earlier procedure time), the application may determine that the first prep dose should be started at 4:00 P.M. the day before the procedure. The application may also determine that the second prep dose should be started at 12:00 A.M. the day of the procedure.

In the event the patient has elected Suprep, GoLytely, or Miralax, and the procedure time is an afternoon session (i.e., 12:00 P.M. or later procedure time), then the application may determine that the first prep dose should be started at 6:00 P.M. the day before the procedure. The application may also determine that the second prep dose should be started at 6:00 A.M. the day of the procedure.

If the application determines that the patient has selected Clenpiq as their desired prep type, the application may determine that the first prep dose should be started between 5:00 P.M. and 9:00 P.M. the day before the procedure, and the second prep dose should be started 5 hours prior to the check-in time on the day of the procedure. In some embodiments, the application may omit checking the procedure time before issuing the instructions to the patient.

If the patient selects Flex Sig for their bowel prep, the application may determine that a day before the procedure the first prep dose consisting of two bottles of Magnesium Citrate should be consumed at 5:00 P.M., and the first enema should be performed at bedtime. The application may also determine that the second enema should be taken at 6:00 A.M. the day of the procedure.

If the user has indicated that they are using Suprep, GoLytely, or Miralax, and have also indicated “Yes” to the two day bowel prep question, the application may automatically determine the required timing and dosing for the patient, and may provide the timing and dosing information to the patient. For instance, the application may determine that the first prep dose for the two day bowel prep should begin at 6:00 P.M. two days prior to the procedure. The application may also determine that the second prep dose for the two day bowel prep should begin at 6:00 A.M. the day prior to the procedure.

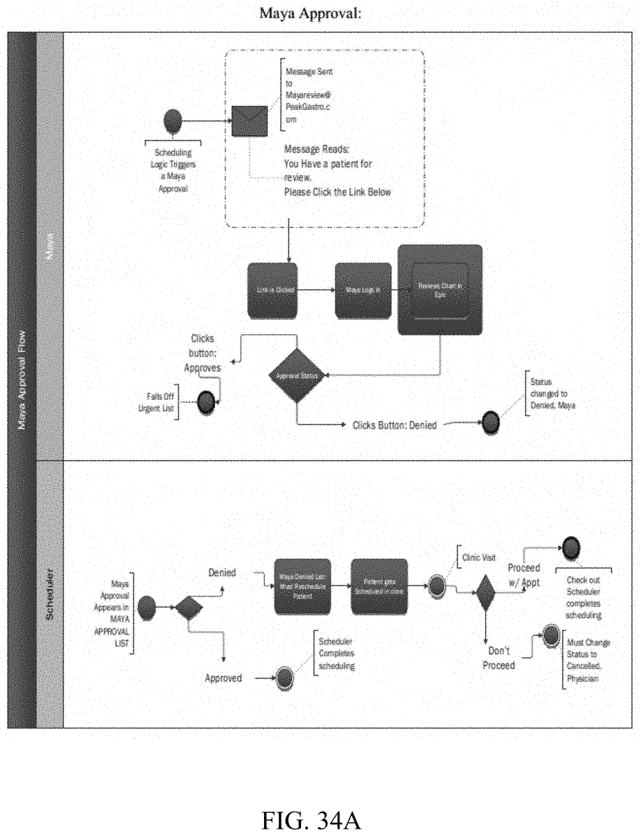

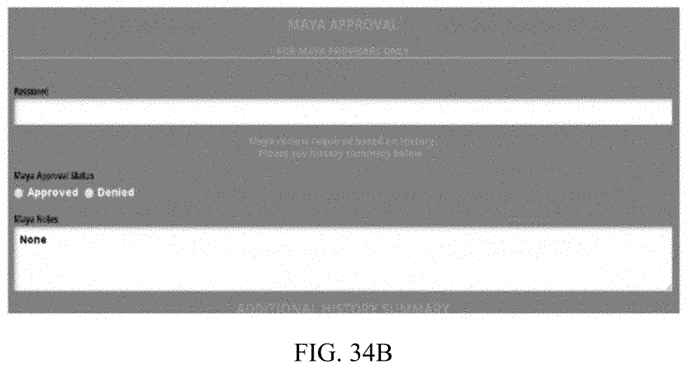

Referring to FIG. 34A, the application may require Maya Approval to permit the scheduling of the encounter. Maya may provide anesthesia for any procedures occurring during an encounter. The Maya Approval workflow may be described with reference to the “Maya Approval” flowchart shown in FIG. 34A.

The Maya Approval Flow flowchart may begin with Maya Approval. The workflow may begin when certain patient answers obtained from the database trigger a need for Maya Approval. The application may conduct a logic check based on the data structures in the database for one or more patient answers that require Maya Approval, and may trigger Maya Approval if any answers require Maya Approval. For instance, if the user enters a “Yes” to any of the questions regarding patient pulmonary/anesthesia events or complications (i.e. history of difficult airway, history of malignant hyperthermia, any supplemental oxygen, new onset shortness of breath, difficulty walking up a flight of stairs, obstructive sleep apnea, cervical neck fusion/ limited neck mobility, pulmonary hypertension, and phentermine), Maya Approval may be required. Moreover, additional responses to patient questions regarding cardiac history and/or current cardiac testing may trigger the Maya Approval workflow system. For instance, if the user indicates a “Yes” to questions regarding history of heart attack and/or heart valve surgery, as well as the echo test, the stress test, and/or the Holter monitor, Maya Approval may be required. Maya Approval may additional or alternatively be required for certain Body Mass Index (BMI) measurements; for instance, a BMI greater than 42 may trigger the need for Maya Approval. An example logic check for the need for Maya Approval can be seen in the Drawings.

In some embodiments, the application may determine the patient answers stored in the database for one or more of the above-mentioned responses that may trigger Maya Approval. Upon determining that the logic requires Maya Approval, the application may dynamically update the label associated with the encounter to the “Scheduled—Maya Approval Pending” label.

Referring to FIG. 34B, in some embodiments, Maya Approval may require a minimum number of days to complete. Accordingly, the application may prevent the scheduler from scheduling the encounter too closely to the date from which it is determined that Maya Approval is required. For example, Maya Approval may take a minimum of two days to complete, and the application may accordingly prevent the scheduler from scheduling the encounter for the patient within two days.

Once Maya Approval has been triggered, the workflow may continue, and the application may send a communication to a Maya approver. In some embodiments, the communication may take the form of an email to the Maya approver. The email may contain a link to the encounter requiring the Maya Approval. The Maya approver may then follow the link to access information related to the encounter through the application. The application may provide the Maya approver with a user interface to conduct the Maya Approval. The user interface may be shown in the image of FIG. 34B. In some embodiments, the user interface may only be visible to Maya members authorized to conduct Maya Approval. In some embodiments, user interface may only be visible to Maya members who have received the communication from the application. The application may provide the Maya approver a location to enter a password. The application may deny entry to any user who does not enter an approved password. In some embodiments, the application may only accept a standardized password known to only the Maya members authorized to conduct Maya Approval.

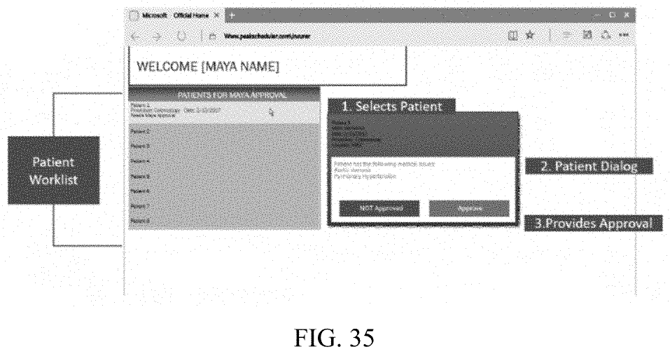

Once the Maya approver has entered the password, the application may allow the Maya approver access to information required to determine approval. An example patient history interface is shown in FIG. 35.

The Maya approver may then view the information and determine whether to approve the procedure. The Maya approver may be presented with a binary choice between “approved” or “NOT approved”. In some embodiments, the Maya approver may be able to enter notes about the procedure and/or reasons for approving or not approving the procedure through the user input device. The application may record the Maya approver choice in the database. In some embodiments, the approval of the encounter may cause a 1 to be stored in a data structure associated with the Maya Approval in the database. The denial of the encounter may cause a 0 to be stored in the data structure associated with the Maya Approval in the database.

The workflow may continue to a step for determining the approval status. In the event that the procedure is approved, the application may verify the location, the date, and the time of the procedure, and may update the encounter interface shown to the scheduler to reflect the Maya approval. For instance, the application may automatically remove the encounter from an urgent list. If the procedure is denied, the application may automatically update the encounter to indicate that the Maya approver has denied the procedure.

The Maya Approval Flow flowchart, executable by one or more processing elements of a computing device, may illustrate the application workflow to display the results of the Maya Approval to the scheduler. The application may access the data structure associated with Maya Approval, and may provide an indicator to the scheduler that indicates whether the Maya approver has granted or denied approval. Upon the entry of approval or denial by the Maya approver, the application may automatically update the label associated with the encounter. For instance, the application may change the label from “Scheduled—Maya Approval Pending” to “Scheduled—Done, Maya Approved” in the event the encounter has been approved. If the encounter is denied, the application may change the label from ““Scheduled—Maya Approval Pending” to “In Progress—Maya Denied” the scheduler may automatically determine whether the entry is an approval or a denial. If the Maya approver denies the encounter, the scheduler may reschedule the patient, and may schedule a clinic visit. The application may then update the label associated with the encounter to “Clinic Visit Needed.” The clinic visit may be scheduled by the scheduler, and may allow a medical professional to evaluate the patient. Upon completing the clinic visit, the medical professional may authorize continuing with the encounter, or may recommend against proceeding. The scheduler may receive the decision of the medical professional, and respond accordingly. In the event the medical professional authorizes the encounter, the scheduler may complete the scheduling. If the medical professional does not authorize the encounter, the scheduler may update the status of the encounter to a “Cancelled—Physician” label to indicate that the encounter is not authorized to proceed.

In some embodiments, the Maya Approval may only be required for certain locations. In one embodiment, the Maya Approval may only be required for an encounter occurring at the FREC or SCOPE locations.

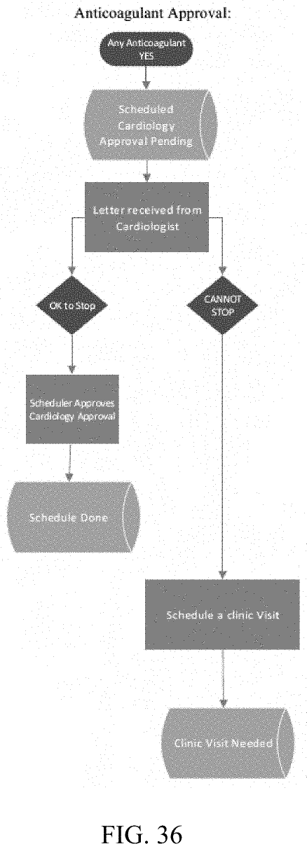

As previously noted, the application may determine that the patient has one or more anticoagulants associated therewith. The application may automatically begin an Anticoagulant Approval workflow upon determining that the patient uses one or more anticoagulants. The workflow for the anticoagulant approval is illustrated as the “Anticoagulant Approval” flowchart of FIG. 36, executable by one or more processing elements.

Upon identification that one or more of the anticoagulants is currently being used by the patient, the application may schedule a cardiology approval. The application may reflect this to the user by displaying an indicator that cardiology approval is required. The scheduler may schedule the encounter with the “Scheduled—Cardiology Approval Pending” label. The application may then query a cardiologist to determine whether the patient is approved to stop using the anticoagulant medication. Based on the received feedback from the cardiologist, the application may adjust the procedure. For instance, if the cardiologist determines that the patient is approved to stop using their medication, the scheduler may mark the encounter as cardiologist approved. In one embodiment, the scheduler may label the encounter with the “Schedule—Done” label to indicate that no further action is required for the encounter. If the cardiologist determines that the medication cannot be stopped, the scheduler may require the patient to schedule a clinic visit. The scheduler may then update the encounter with the “Clinic Visit Needed” label to indicate that the patient may need to schedule a clinic visit to proceed with the encounter.

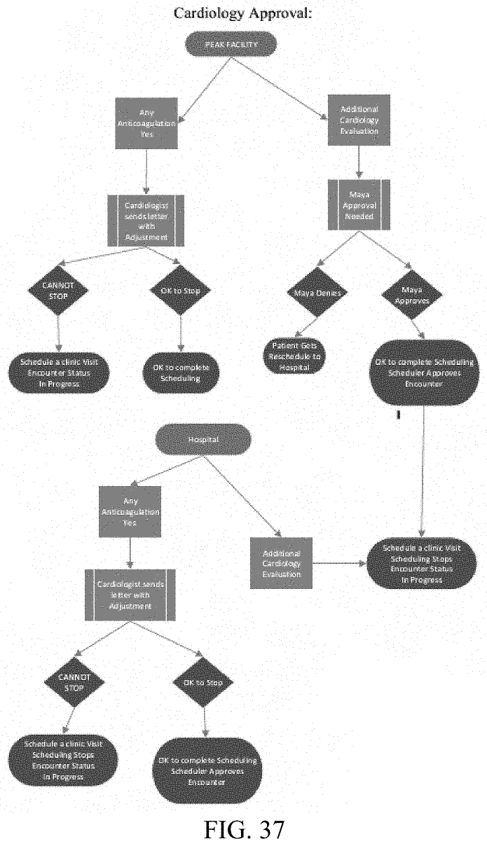

In some embodiments, the cardiology approval may be different for a hospital encounter than for a non-hospital encounter. A possible cardiology approval is described herein with reference to a “Cardiology Approval” flowchart illustrated in FIG. 37, executable by one or more processing elements.

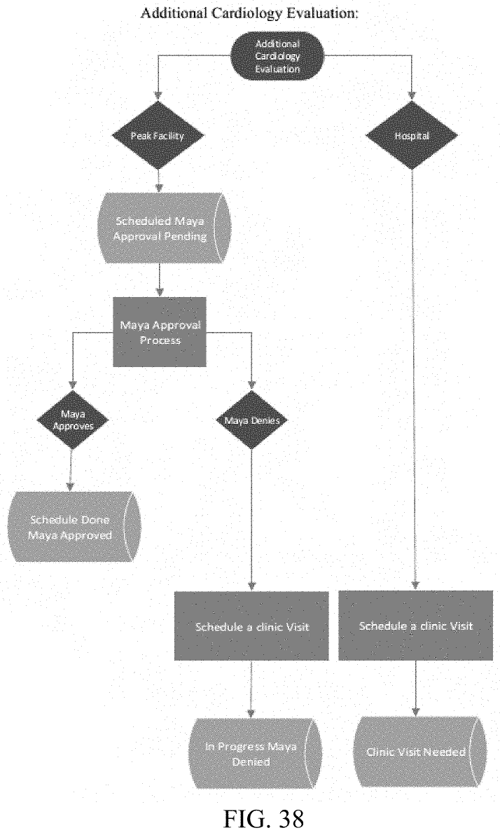

With regard to non-hospital approval, such as at the Peak facilities, the Cardiology Approval flowchart of FIG. 37 may begin by determining if anticoagulant approval is required, as noted with respect to the “Anticoagulant Approval” flowchart of FIG. 36. Additionally or alternatively, the application may determine if additional cardiology evaluation is required. The application may do this in a variety of ways, such as by checking the stored data structures on the database to determine if the patient indicated any additional cardiac testing and/or evaluation. The additional cardiology evaluation may be described with reference to an “Additional Cardiology Evaluation” flowchart shown in FIG. 38, executable by one or more processing elements.

The application may first determine whether the encounter is to occur in the peak facility or a hospital. Upon determination that the encounter is to occur in a hospital, the application may automatically require that the patient schedule a clinic visit. The application may then adjust the label associated with the encounter from the “In Progress” label to the “Clinic Visit Needed” label.

At the peak facility, the application may then determine whether Maya Approval is needed. The application may update the label associated with the encounter to the “Scheduled—Maya Approval Pending” label. The application may proceed through the “Maya Approval” flowchart to determine whether Maya approves the encounter. In the event that Maya approves the encounter, the application may update the label associated with the encounter to the “Scheduled—Done, Maya Approved” label. If Maya denies the encounter, the application may update the label associated with the encounter to the “In Progress—Maya Denied” label to indicate that a clinic visit is needed so a medical professional may determine the next steps.

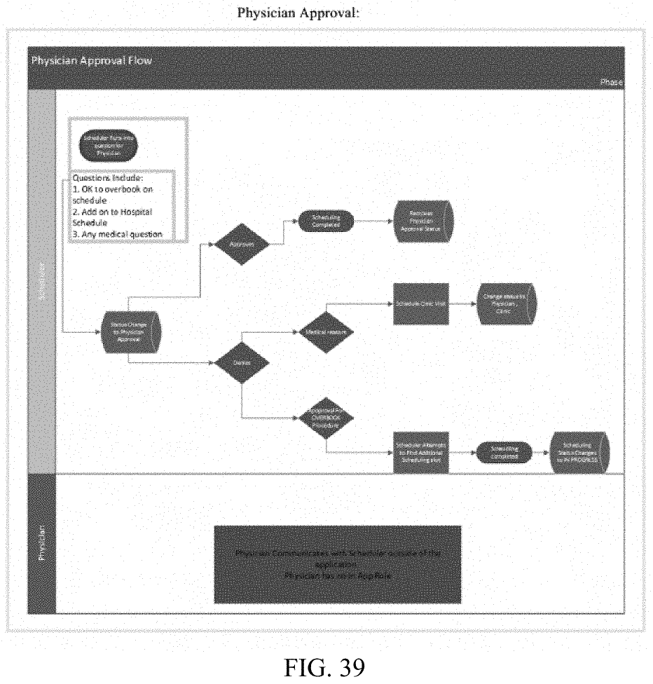

The application may determine that physician approval is needed. The application may then implement a physician approval workflow. The workflow may be described with reference to the “Physician Approval” flowchart shown FIG. 39 executable by one or more processing elements. As detailed in the “Physician Approval” flowchart, the scheduler may encounter an issue that requires physician input. Some non-limiting examples may include whether it is acceptable to overbook a certain day on the physician's schedule, whether it is acceptable to add on to the hospital schedule, or any other medical question the physician is qualified to answer.

Accordingly, the application may update the label associated with the encounter to a “Physician Approval” label to indicate that the encounter is subject to physician approval and/or input. In some embodiments, the application may then prompt the scheduler to communicate with the physician to receive physician input. In some embodiments, the physician may communicate with the scheduler outside of the application. In some embodiments, the physician may not input any information into the application. Once the scheduler has information regarding the physician's approval and/or input, the scheduler may enter the information into the application. If the physician approves the encounter, the application may update the label associated with the label to the “Scheduled—Done” label to indicate that the encounter is approved. If the physician denies the encounter, the application may provide a textbox for the scheduler to enter notes from the physician about the reasons for the denial, and may update the label associated with the encounter to the “Clinic Visit Needed” label to indicate that a clinic visit is needed to determine the next steps.

In some embodiments, the scheduler may have inquired about overbooking. In one embodiment, the physician denial of the overbooking may be entered by the scheduler into the application. The scheduler may attempt to find a different scheduling date, time, and/or location. The application may automatically, upon the denial of the overbooking, update the label associated with the encounter to the “In Progress” label.

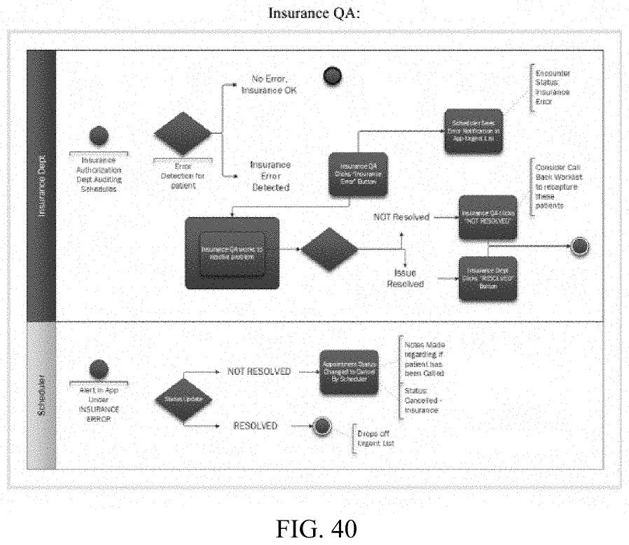

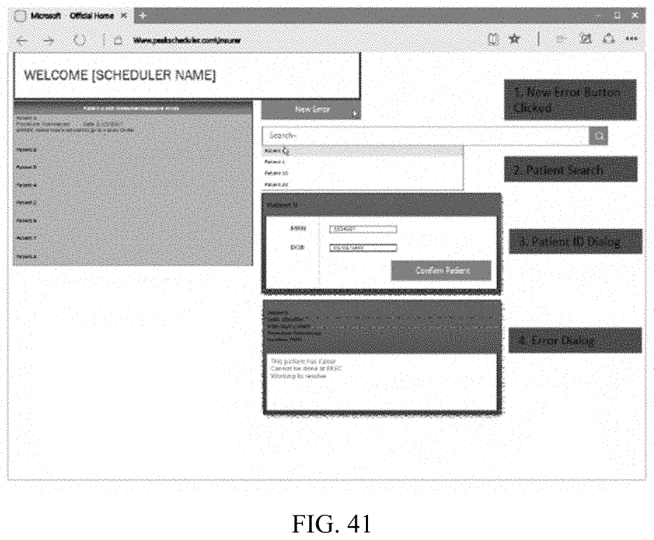

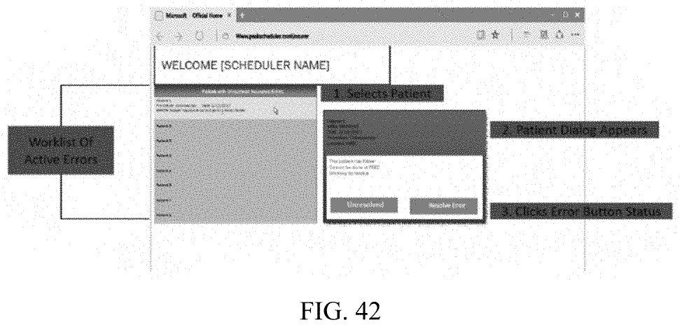

The application may be designed to communicate with the insurance provider and provide a quality assurance workflow. The quality assurance workflow may be shown in the “Insurance QA” flowchart of FIG. 40, executable by one or more processing elements. An insurance authorization department may provide insurance authorization for the patient. The insurance authorization department may conduct an audit of the schedules associated with its patients. Upon detection of an error in the insurance of the patient, the application may automatically receive an error notification and may automatically indicate to the scheduler that there is an insurance error associated with the encounter. The application may then add the encounter to an urgent list. Example scheduler interfaces for the insurance error may be seen in the images of FIGS. 41-42.

Upon further review, the insurance authorization department may conclude that there is no error in the insurance of the patient, and may send an indicator to the application indicating that the error has been resolved and the application may automatically remove the encounter from the urgent list and update the label associated with the encounter.

If the insurance authorization department determines there is an error in the insurance for the patient, an insurance quality assurance (QA) agent may indicate that there is an error by selecting an insurance error button. Once the QA agent selects the insurance error button, the QA agent may work to resolve the problem with the insurance of the patient, and the application may automatically update the label associated with the encounter to an “Insurance Error” label, to indicate that the QA agent has viewed the encounter and determined an error with the insurance of the patient. After the QA agent has worked to resolve the problem, the QA agent may report that the issue is either resolved or not resolved. The QA agent may report the issue is resolved by selecting a resolved button. When the QA agent selects the resolved button, the issue may be considered resolved, and the application may automatically remove the encounter from the urgent list and update the label associated with the encounter. The QA agent may determine that the issue is not resolved, and may select a not resolved button. When the QA agent selects the not resolved button, the issue may be considered not resolved, and the application may automatically update the label associated with the encounter to a “Cancelled—Insurance” label to indicate that an issue with the insurance resulted in a cancelled encounter.

The application may present the patient with dynamic instructions to follow based on the stored data structures associated with the patient responses and any approval workflows. The description of the instructions may be given with reference to the “Procedure Information” layout and the “Example Instructions” in the Drawings.

The instructions may contain conditional outputs, and may update depending on any one of the approvals previously discussed. A first page of the instructions may indicate procedural and/or patient information, such as patient name, patient DOB, patient procedure type, the physician overseeing the procedure, the date, time, and location of the procedure, and the procedure check-in time. The application may construct the instructions by accessing the data structures of the database and, through the use of the processor, update the instructions to contain the patient and encounter information.

The instructions may contain a section indicating that the Maya Approval is currently pending approval, and that the procedure it tentatively scheduled. This may correspond to the Scheduled—Maya Approval Pending” label associated with the encounter. In some embodiments, the application may determine the data structure associated with Maya Approval, and may update the instructions upon Maya approval or denial of the encounter. The application may then update the instructions, causing the instructions to display updated instructions for the patient. In the event that the Maya approver approved the encounter, the instructions may indicate that the procedure is scheduled. If the Maya approver denied the encounter, the instructions may indicate to the patient that a clinic visit is required.

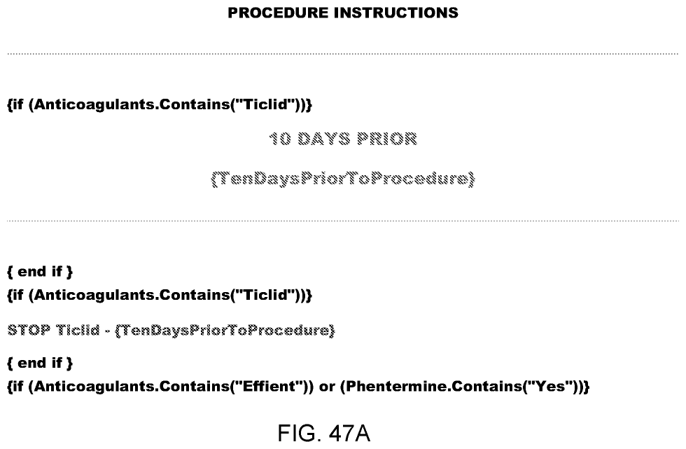

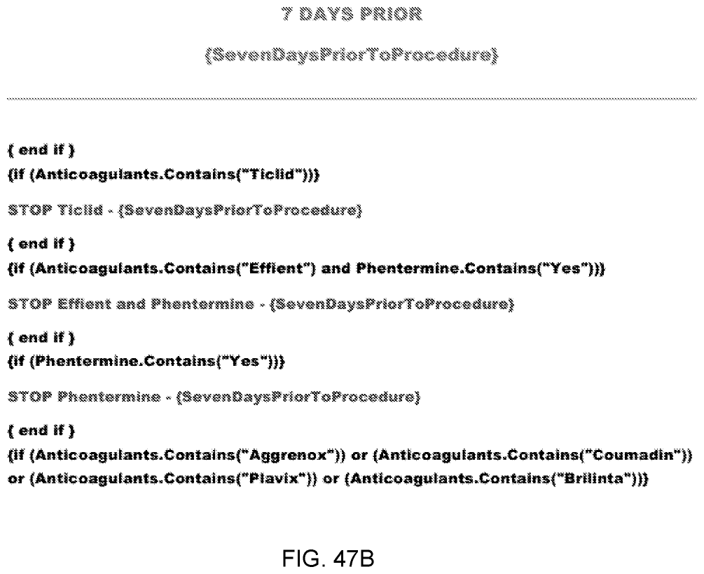

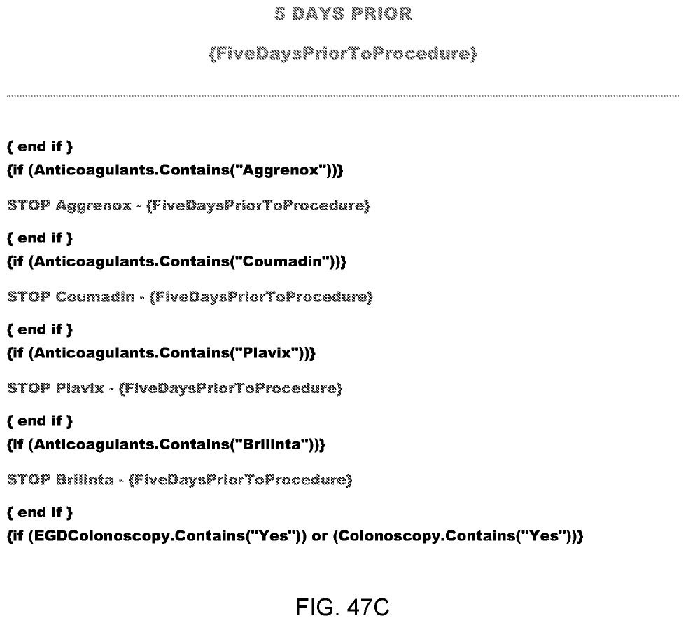

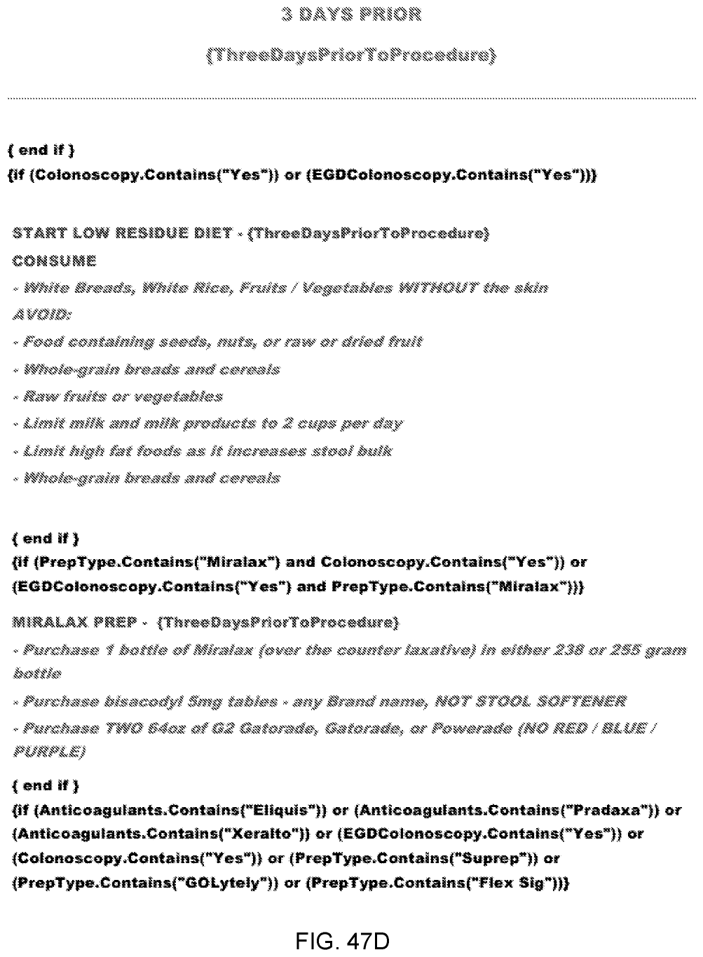

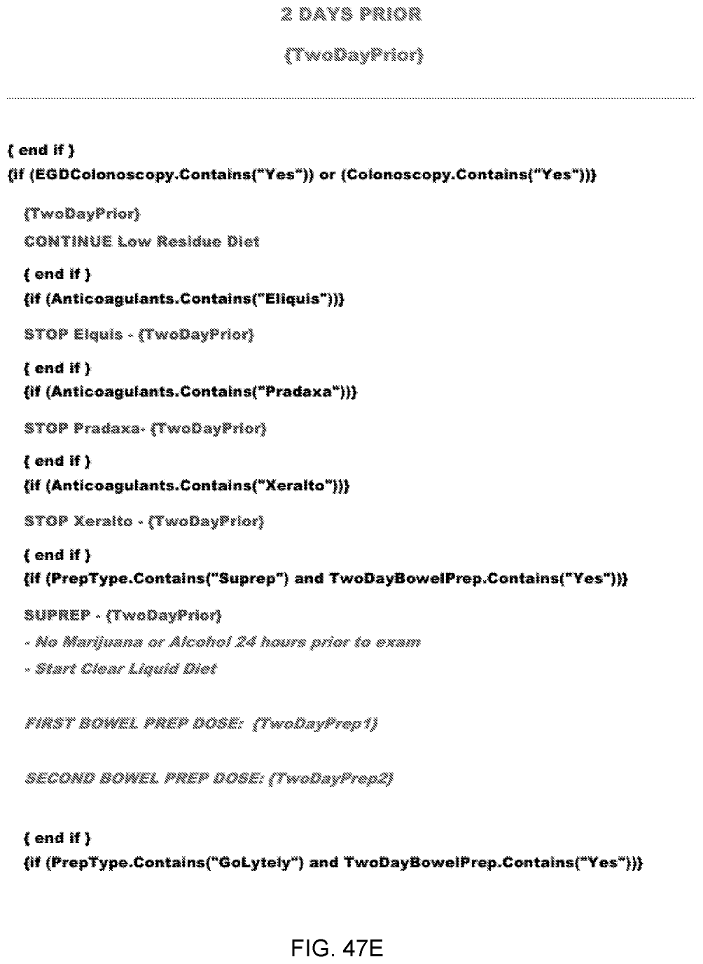

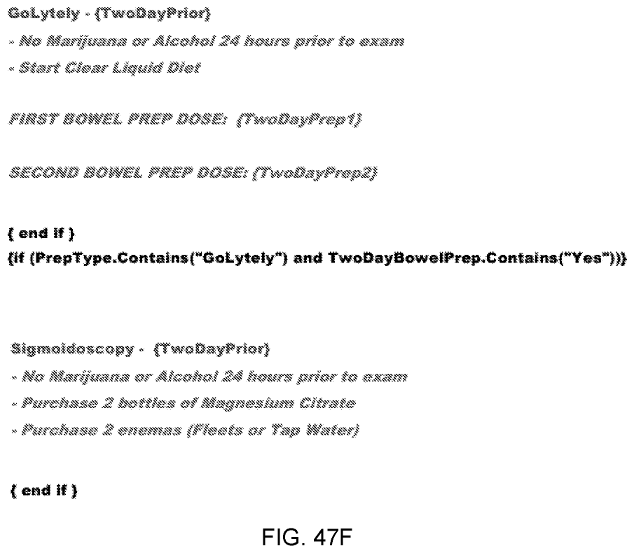

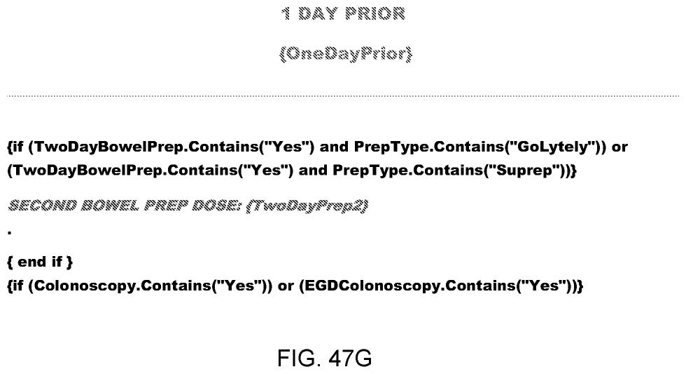

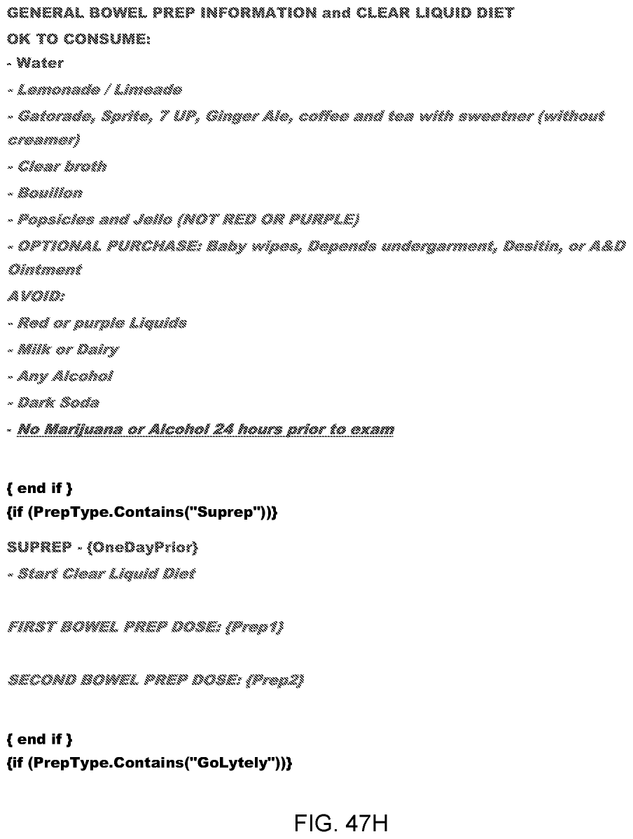

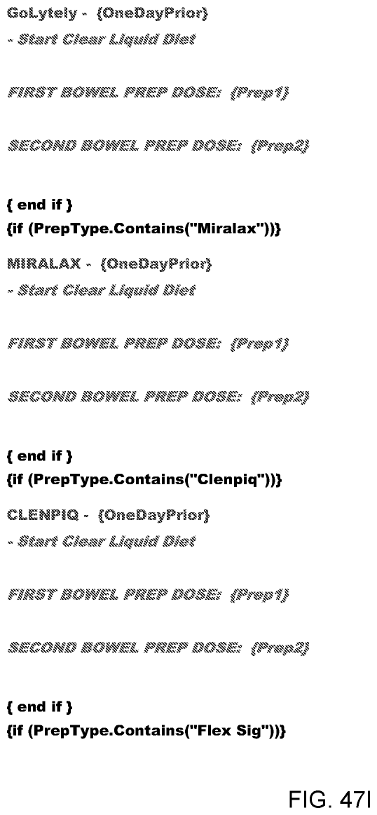

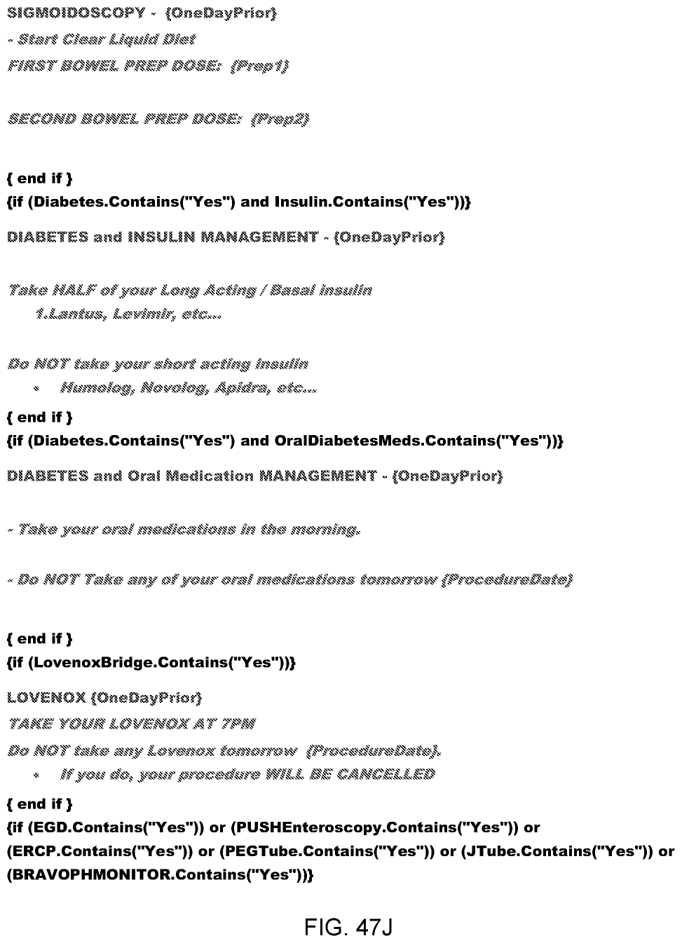

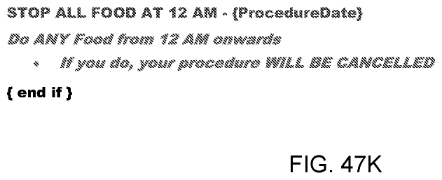

The instructions may include procedure instructions, which may guide the user through any preparation required for the procedure. For instance, Ticlid is typically stopped ten days prior to the procedure. The application may access, through a processor, the patient response to anticoagulant use. If the patient indicated that they take Ticlid, then application may update the procedure instructions to indicate that the patient should stop the use of Ticlid ten days prior to the procedure. In some embodiments, as shown in the Drawings (e.g., FIG. 47A), the application may construct a heading “10 Days Prior” with the instructions to halt the use of Ticlid thereunder. In some embodiments, the heading may only be constructed if the patient response indicated Ticlid use.

The procedure instructions may further comprise headings to display to the patient conditioned on patient responses to the questions posed in the application. For instance, in the event the patient indicated they take Effient or phentermine, the application may construct a “7 Days Prior” heading in the procedure instructions with instructions thereunder. This may be because the patient must stop the use of Effient and phentermine seven days before the procedure to limit the chance of complications during the procedure. The application may then access the database through the processor to determine a series of conditional logics to determine what to display to the user. For instance, if the patient indicated that they take both Effient and phentermine, the application may update the procedure instructions to instruct the user to halt the use of both Effient and phentermine. In the event that the patient indicated they take only Effient or phentermine, the procedure instructions may reflect this information by instructing the user to stop taking the Effient or phentermine, respectively.

The application may further check through a processor the patient responses for their use of Aggrenox, Coumadin, Plavix, and Brilinta. Aggrenox, Coumadin, Plavix, and Brilinta are typically stopped five days prior to the procedure, to reduce the risk of procedural complications. Accordingly, if the application detects that the patient takes any of these medications, the application may instruct the procedure instructions to generate a “5 Days Prior” heading with instructions thereunder. Upon the generation of the “5 Days Prior” heading, the application may then conditionally determine if the patient takes Aggrenox, Coumadin, Plavix, or Brilinta, and instruct the patient to stop the use thereof through the procedure instructions. In some embodiments, the application may check each anticoagulant individually, and present a separate instruction to stop the use of the medication for each of the medications that the patient has indicated they use.

The application may further check through the processor whether the patient is scheduled for either a colonoscopy or a combination of the EGD and the colonoscopy. In the event the patient has indicated that either procedure will be needed during the encounter, the application may generate a “3 Days Prior” heading in the procedure instructions along with additional instructions for the colonoscopy thereunder. The procedure instructions may inform the patient any changes to diet required for the colonoscopy procedure. For instance, the procedure instructions may indicate that the patient should begin a low residue diet by consuming white breads and rice, and skinless fruits and vegetables; the procedure instructions may instruct the user to avoid foods containing seeds, nuts, or raw or dried fruit, whole-grain breads and cereals, raw fruits or vegetables. The procedure instructions may further instruct the patient to limit certain foods, such as limiting milk and milk products to 2 cups per day, as well as limiting high fat foods.

The application may also conditionally determine, through the processor, whether the patient has selected a prep plan associated with the colonoscopy. For instance, in the event the patient has selected “Miralax” for their prep plan, the procedure instructions may provide instructions on the use thereof under the “3 Days Prior” heading. For instance, the procedure instructions may instruct the user to purchase a bottle of Miralax, bisacodyl tables, and two 64 oz of Gatorade or Powerade. In some embodiments, the procedure instructions may limit the type of Gatorade (e.g., the instructions may indicate that the color may not be red, blue, or purple).

The application may conditionally determine, through the processor, any requirements for two days prior to the procedure, and may generate a “2 Days Prior” heading in the procedure instructions containing instructions for the patient to conduct two days prior to the date of procedure. The application may check multiple conditions associated with patient input to determine if the “2 Days Prior” heading is required. The application may check whether the patient indicated that they take Eliquis or Pradaxa, whether the patient indicated that they take Xeralto or require a combination of the EGD and the colonoscopy, whether the patient requires a colonoscopy or they plan on using Suprep for their prep plan, and whether the patient plans on using GoLytely or Flex Sig for their prep plan. If any of the above are true, the application may instruct the procedure instructions to present the “2 Days Prior” heading to the patient along with instructions.

Under the “2 Days Prior” heading, the application may conduct additional conditionality checks. For instance, the procedure instructions may instruct the patient to continue the low residue diet if the application detects that the patient is receiving the colonoscopy or the combination of the EGD and the colonoscopy. Additionally, the procedure instructions may determine if the patient has indicated any use of Eliquis, Pradaxa, and/or Xeralto. If any of these anticoagulants are utilized by the patient, the procedure instructions may instruct the patient to discontinue use thereof. The application may determine if the patient has selected Suprep as their prep plan and has elected to do the two day bowel prep. If both conditions are true, the procedure instructions may provide instructions to the patient. For instance, the procedure instructions may inform the user that no marijuana or alcohol may be consumed 24 hours prior to the procedure, and that the user should begin a liquid diet. The procedure instructions may also instruct the patient the times to conduct the first and second bowel prep doses.

The application may determine if the patient has selected GoLytely as their prep plan and elected the two day bowel prep. If both conditions are true, the procedure instructions may provide instructions to the patient. For instance, the procedure instructions may inform the user that no marijuana or alcohol may be consumed 24 hours prior to the procedure, and that the user should begin a liquid diet. The procedure instructions may also instruct the patient to purchase 2 bottles of magnesium citrate, and two enemas.

The procedure instructions may provide a “1 Day Prior” heading with instructions thereunder, based on conditional logic checked by the application. In some embodiments, the “1 Day Prior” heading may be provided regardless of patient input. The application may then conditionally determine which instructions are required by the patient based on inputted data. For instance, the application may check whether the patient has elected the two day bowel prep. If the patient has elected the two-day bowel prep, the application may determine whether or not the patient elected either the GoLytely or the Suprep for their prep plan. If the patient elected either the GoLytely or the Suprep, the procedure instructions may provide the user with information regarding the second bowel prep dose. The application may check whether the patient indicated they require the colonoscopy or the combination of the EGD and the colonoscopy. If true, the procedure instructions may provide the user with general bowel prep information and a clear liquid diet.

The clear liquid diet may be a diet designed to optimize the efficiency and effectiveness of the colonoscopy, and may instruct the patient as to appropriate and inappropriate food and drink. For instance, the procedure instructions may indicate that the patient is OK to consume water, lemonade, limeade, Gatorade, coffee and tea with sweetener, clear broth, bouillon, popsicles, Jell-O, and clear sodas. In some embodiments, the procedure instructions may indicate that red or purple popsicles and Jell-O are prohibited. The procedure instructions may indicate what is inappropriate for the clear liquid diet, such as any red or purple liquids, any milk or diary, any alcohol, any dark soda, and any marijuana or alcohol 24 hours prior to the exam may be prohibited.

Additionally or alternatively, the application may determine which bowel prep the patient selected. For instance, the procedure instructions may determine whether the user selected Suprep, GoLytely, Miralax, Clenpiq, or Flex Sig. The procedure instructions may then indicate to the user to begin the clear liquid diet as well as the times to take the first and second bowel prep doses.

The application may additionally check whether the patient has entered information pertaining to diabetes and insulin. In some embodiments, the application may, through the processor, determine if the patient has diabetes and insulin therefor. If both conditions are true, the procedure instructions may provide diabetes and insulin management. For instance, the procedure instructions may be updated to instruct the user to take half of the patient's long acting/basal insulin, and to not take short acting insulin. Additionally or alternatively, the application may determine if the patient has diabetes and an oral diabetes medication therefor. If both conditions are true, the procedure instructions may be updated to instruct the user to take their oral medication in the morning, and to not take the oral medication the day of the procedure.

In the event the procedure requires the use of Lovenox bridge, the procedure instructions may provide the user with instructions on the use thereof. For instance, the procedure instructions may instruct the user to take the Lovenox the day before the procedure, and to not take any Lovenox on the day of the procedure at the risk of cancelling the procedure.

The procedure instructions may provide further instruction for any other procedure or add-on. For instance, the application may, through the processor, determine if the user requires the EGD, the PUSH Enterscopy, the ERCP, the PEG Tube, the J-tube, or the pH monitor. If any of these are detected by the application, the procedure instructions may generate instructions that prompt the patient to stop all food at 12 AM and any food from 12 AM onwards. In some embodiments, the procedure instructions may indicate that failing to comply with this order will result in the cancellation of the procedure.

The procedure instructions may provide instructions for the day of the procedure. The procedure instructions may generate a “Day of Procedure” heading with corresponding instructions to the patient based on conditional inputs from the patient. In the event the patient has scheduled the colonoscopy or the combination of the EGD and the colonoscopy, the procedure instructions may instruct the user to take the second bowel prep dose. The procedure instructions may indicate that the patient is to arrive at the scheduled check-in time. The procedure instructions may access the check-in time stored in the database and display the check-in time to the user.

The procedure instructions may give general, non-dynamic instructions to the patient on for the day of the procedure. For instance, the non-dynamic instructions may inform the user that the patient must have a responsible adult therewith for the entirety of the day of the procedure. The instructions may indicate that the user is not to take any blood thinners, unless directed to do so by a physician. The instructions may instruct the user to not consume any food/drinks/medications 4 hours prior to the procedure, and that no marijuana or alcohol should be consumed 24 hours prior to the procedure.

The non-dynamic instructions may indicate that a user with an afternoon appointment should be available for the entirety of the day, and the afternoon appointment may be subject to a change to a morning appointment upon any morning appointment cancellations. The non-dynamic instructions may be given to the user for one or more of the procedures previously mentioned.

The application may determine whether the patient was directed to follow a Lovenox bridge program. If the application determines that the patient followed the Lovenox bridge, the procedure instructions may be updated to provide the user with instructions to not take the Lovenox.

The procedure instructions may provide instructions to the patient regarding diabetes and insulin management. For instance, the application may determine whether the user has diabetes and whether the patient uses insulin and/or oral medication. The procedure instructions may then instruct the patient to not take any insulation and/or any oral medication.

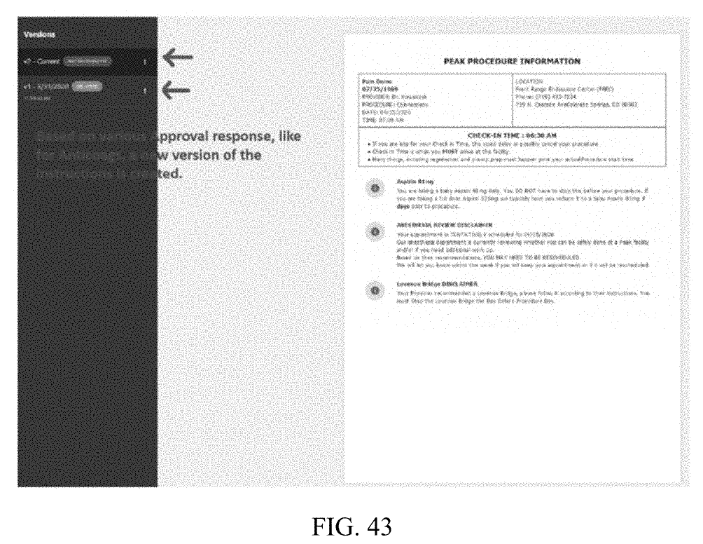

Additionally or alternatively, the application may provide new versions of instructions to the patient based on one or more conditional approvals, and may indicate that the instructions previously provided may have changed based on the one or more conditional approvals. The application may create a new version of the instructions and send the patient the new version of the instructions. The application may notify the scheduler as to the change in the one or more conditional approvals, as well as to the status of the instructions as shown in the image of FIG. 43.

As seen in the image of FIG. 43, the application may provide the user interface where the scheduler may view previous versions of the instructions sent to the patient as well as a the most current set of instructions. For example, the scheduler may see that there is a first set of instructions called “v1”, which was delivered to the patient on Mar. 31, 2020. However, the first set of instructions may be subject to the one or more conditional approvals, and may be updated to a second set of instructions called “v2”, which may contain changes based on the results of the one or more conditional approvals. The application may provide one or more labels to indicate the delivery status to the patient. As shown, the first set of instructions has been delivered to the patient, as indicated by a “Delivered” label associated with the instructions. The second set of instructions is tagged with a “Not Delivered Yet” label, to indicate that the second set of instructions has not been sent to the patient.

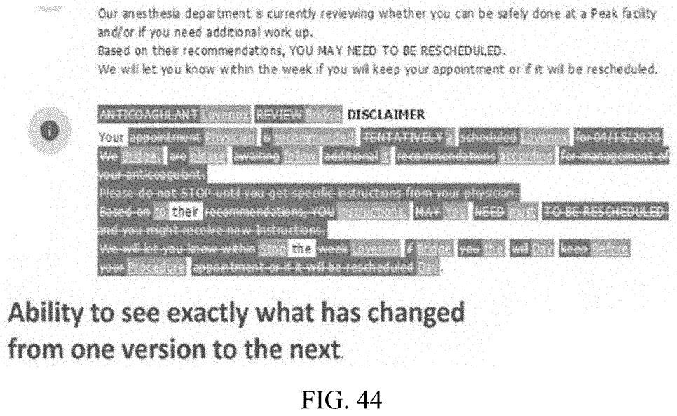

The second set of instructions may contain changes that instruct the patient differently than the first set of instructions. The changes may be related to any of the patient instructions already described above, and may be dependent on the one or more conditional approvals. FIG. 44 illustrates an image depicting an exemplary change from a portion of the first set of instructions to the second set of instructions.

As noted, the application may update the instructions given to the patient. In the subject image, the first version of the instructions given to the patient may indicate that physician approval for anticoagulant use. The instructions in this example may provide an anticoagulant review disclaimer that states “Your appointment is TENTATIVELY scheduled for Apr. 15, 2020. We are awaiting additional recommendations for management of your anticoagulant. Please do not STOP until you get specific instructions from you physician. Based on their recommendation, YOU MAY NEED TO BE RESCHEDULED and you might receive new Instructions. We will let you know within the week if you will keep your appointment or if it will be rescheduled.”

After providing the first version of the instructions to the patient, the application may conduct conditional checks on one or more of the approvals previously mentioned. Upon a change to any approval status, as determined by the application by accessing the data structures in the database by the use of the processor, the application may generate changes to the instructions, and issue a new set of the instructions to the patient. As noted in the example image of FIG. 44, the physician may have approved the Lovenox bridge to provide the patient with anticoagulants prior to the encounter. The physician approval of the Lovenox bridge may permit the application to automatically determine, based on the data structure in the database associated with the Lovenox bridge, that the instructions need updating, and that the new set of instructions should be sent to the patient. The application may then indicate to the scheduler that the new set of instructions is required. The application may then send the new set of instructions to the patient. The subject example of the Lovenox bridge is in no way limiting, and any change may result in the application issuing the new set of instructions. Any change may include, but is in no way limited to, any of the patient responses to any questions or medical professional approvals previously mentioned.

As shown in the image of FIG. 44, the new set of instructions changes the anticoagulant review disclaimer to a Lovenox bridge disclaimer that states “Your Physician recommended a Lovenox Bridge, please follow it according to their instructions. You must stop the Lovenox Bridge the Day Before Procedure Day.” The new instructions may allow the patient to know that the physician approved the encounter, and that the Lovenox bridge is to be taken instead of the anticoagulants currently used by the patient. In some embodiments, the application may provide a tracked changes format so the patient may view the changes to the instructions. The scheduler and/or the patient may be able to see the exact changes made to the instructions. The additions may be indicated in green, with the deletions shown in red.

Any of the steps, functions, and operations discussed herein can be performed continuously and automatically.

The exemplary systems and methods of this disclosure have been described in relation to adaptable instructions for a patient procedure. However, to avoid unnecessarily obscuring the present disclosure, the preceding description omits a number of known structures and devices. This omission is not to be construed as a limitation of the scope of the claimed disclosure. Specific details are set forth to provide an understanding of the present disclosure. It should, however, be appreciated that the present disclosure may be practiced in a variety of ways beyond the specific detail set forth herein.

Furthermore, while the exemplary embodiments illustrated herein show the various components of the system collocated, certain components of the system can be located remotely, at distant portions of a distributed network, such as a LAN and/or the Internet, or within a dedicated system. Thus, it should be appreciated, that the components of the system can be combined into one or more devices, such as a server, communication device, or collocated on a particular node of a distributed network, such as an analog and/or digital telecommunications network, a packet-switched network, or a circuit-switched network. It will be appreciated from the preceding description, and for reasons of computational efficiency, that the components of the system can be arranged at any location within a distributed network of components without affecting the operation of the system.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire, and fiber optics, and may take the form of acoustic or light waves, such as those generated during radio-wave and infra-red data communications.

While the flowcharts have been discussed and illustrated in relation to a particular sequence of events, it should be appreciated that changes, additions, and omissions to this sequence can occur without materially affecting the operation of the disclosed embodiments, configuration, and aspects.

A number of variations and modifications of the disclosure can be used. It would be possible to provide for some features of the disclosure without providing others.

In yet another embodiment, the systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of this disclosure. Exemplary hardware that can be used for the present disclosure includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

In yet another embodiment, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or VLSI design. Whether software or hardware is used to implement the systems in accordance with this disclosure is dependent on the speed and/or efficiency requirements of the system, the particular function, and the particular software or hardware systems or microprocessor or microcomputer systems being utilized.

In yet another embodiment, the disclosed methods may be partially implemented in software that can be stored on a storage medium, executed on programmed general-purpose computer with the cooperation of a controller and memory, a special purpose computer, a microprocessor, or the like. In these instances, the systems and methods of this disclosure can be implemented as a program embedded on a personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

Although the present disclosure describes components and functions implemented in the embodiments with reference to particular standards and protocols, the disclosure is not limited to such standards and protocols. Other similar standards and protocols not mentioned herein are in existence and are considered to be included in the present disclosure. Moreover, the standards and protocols mentioned herein and other similar standards and protocols not mentioned herein are periodically superseded by faster or more effective equivalents having essentially the same functions. Such replacement standards and protocols having the same functions are considered equivalents included in the present disclosure.

The present disclosure, in various embodiments, configurations, and aspects, includes components, methods, processes, systems and/or apparatus substantially as depicted and described herein, including various embodiments, subcombinations, and subsets thereof. Those of skill in the art will understand how to make and use the systems and methods disclosed herein after understanding the present disclosure. The present disclosure, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease, and/or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments, configurations, or aspects for the purpose of streamlining the disclosure. The features of the embodiments, configurations, or aspects of the disclosure may be combined in alternate embodiments, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the disclosure.

Moreover, though the description of the disclosure has included description of one or more embodiments, configurations, or aspects and certain variations and modifications, other variations, combinations, and modifications are within the scope of the disclosure, e.g., as may be within the skill and knowledge of those in the art, after understanding the present disclosure. It is intended to obtain rights, which include alternative embodiments, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Aspects of the present disclosure may take the form of an embodiment that is entirely hardware, an embodiment that is entirely software (including firmware, resident software, micro-code, etc.) or an embodiment combining software and hardware aspects that may all generally be referred to herein as a “circuit,” “module,” or “system.” Any combination of one or more computer-readable medium(s) may be utilized. The computer-readable medium may be a computer-readable signal medium or a computer-readable storage medium.

A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device. Program code embodied on a computer-readable medium may be transmitted using any appropriate medium, including, but not limited to, wireless, wireline, optical fiber cable, RF, etc., or any suitable combination of the foregoing.

The terms “determine,” “calculate,” “compute,” and variations thereof, as used herein, are used interchangeably and include any type of methodology, process, mathematical operation or technique.

### Computing Device for Implementing Aspects of the System of FIG. 1

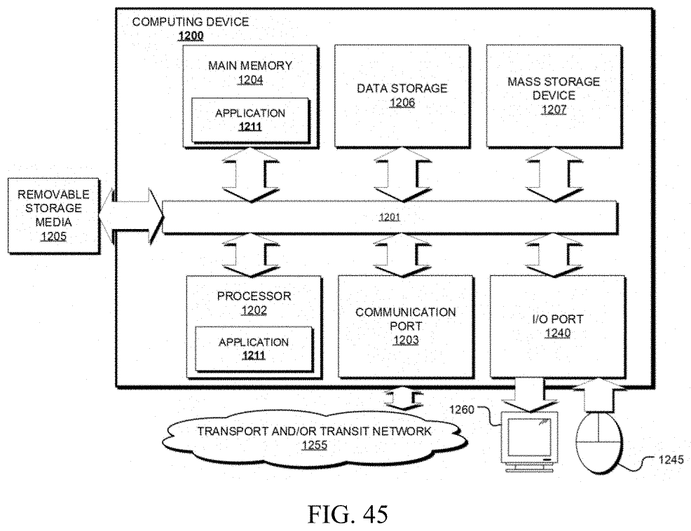

Referring to FIG. 45, a computing device 1200 is illustrated which may be configured, via an application 1211 and/or other software components such as the application shown in FIG. 1 and described herein, to execute functionality for producing dynamically-alterable instructions, which may be translated to software or machine-level code, and may be installed to and/or executed by the computing device 1200 such that the computing device 1200 is configured to compute the dynamically-alterable instructions. It is contemplated that the computing device 1200 may include any number of devices, such as personal computers, server computers, hand-held or laptop devices, tablet devices, multiprocessor systems, microprocessor-based systems, set top boxes, programmable consumer electronic devices, network PCs, minicomputers, mainframe computers, digital signal processors, state machines, logic circuitries, distributed computing environments, and the like.

The computing device 1200 may include various hardware components, such as a processor 1202, a main memory 1204 (e.g., a system memory), and a system bus 1201 that couples various components of the computing device 1200 to the processor 1202. The system bus 1201 may be any of several types of bus structures including a memory bus or memory controller, a peripheral bus, and a local bus using any of a variety of bus architectures. For example, such architectures may include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

The computing device 1200 may further include a variety of memory devices and computer-readable media 1207 that includes removable/non-removable media and volatile/nonvolatile media and/or tangible media, but excludes transitory propagated signals. Computer-readable media 1207 may also include computer storage media and communication media. Computer storage media includes removable/non-removable media and volatile/nonvolatile media implemented in any method or technology for storage of information, such as computer-readable instructions, data structures, program modules or other data, such as RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information/data and which may be accessed by the computing device 1200. Communication media includes computer-readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. For example, communication media may include wired media such as a wired network or direct-wired connection and wireless media such as acoustic, RF, infrared, and/or other wireless media, or some combination thereof. Computer-readable media may be embodied as a computer program product, such as software stored on computer storage media.

The main memory 1204 includes computer storage media in the form of volatile/nonvolatile memory such as read only memory (ROM) and random access memory (RAM). A basic input/output system (BIOS), containing the basic routines that help to transfer information between elements within the computing device 1200 (e.g., during start-up) is typically stored in ROM. RAM typically contains data and/or program modules that are immediately accessible to and/or presently being operated on by processor 1202. Further, data storage 1206 in the form of Read-Only Memory (ROM) or otherwise may store an operating system, application programs, and other program modules and program data.

The data storage 1206 may also include other removable/non-removable, volatile/nonvolatile computer storage media. For example, the data storage 1206 may be: a hard disk drive that reads from or writes to non-removable, nonvolatile magnetic media; a magnetic disk drive that reads from or writes to a removable, nonvolatile magnetic disk; a solid state drive; and/or an optical disk drive that reads from or writes to a removable, nonvolatile optical disk such as a CD-ROM or other optical media. Other removable/non-removable, volatile/nonvolatile computer storage media may include magnetic tape cassettes, flash memory cards, digital versatile disks, digital video tape, solid state RAM, solid state ROM, and the like. The drives and their associated computer storage media provide storage of computer-readable instructions, data structures, program modules, and other data for the computing device 1200.

A user may enter commands and information through a user interface 1240 (displayed via a monitor 1260) by engaging input devices 1245 such as a tablet, electronic digitizer, a microphone, keyboard, and/or pointing device, commonly referred to as mouse, trackball or touch pad. Other input devices 1245 may include a joystick, game pad, satellite dish, scanner, or the like. Additionally, voice inputs, gesture inputs (e.g., via hands or fingers), or other natural user input methods may also be used with the appropriate input devices, such as a microphone, camera, tablet, touch pad, glove, or other sensor. These and other input devices 1245 are in operative connection to the processor 1202 and may be coupled to the system bus 1201, but may be connected by other interface and bus structures, such as a parallel port, game port or a universal serial bus (USB). The monitor 1260 or other type of display device may also be connected to the system bus 1201. The monitor 1260 may also be integrated with a touch-screen panel or the like.

The computing device 1200 may be implemented in a networked or cloud-computing environment using logical connections of a network interface 1203 to one or more remote devices, such as a remote computer. The remote computer may be a personal computer, a server, a router, a network PC, a peer device or other common network node, and typically includes many or all of the elements described above relative to the computing device 1200. The logical connection may include one or more local area networks (LAN) and one or more wide area networks (WAN), but may also include other networks. Such networking environments are commonplace in offices, enterprise-wide computer networks, intranets and the Internet.

When used in a networked or cloud-computing environment, the computing device 1200 may be connected to a public and/or private network through the network interface 1203. In such embodiments, a modem or other means for establishing communications over the network is connected to the system bus 1201 via the network interface 1203 or other appropriate mechanism. A wireless networking component including an interface and antenna may be coupled through a suitable device such as an access point or peer computer to a network. In a networked environment, program modules depicted relative to the computing device 1200, or portions thereof, may be stored in the remote memory storage device.

Certain embodiments are described herein as including one or more modules. Such modules are hardware-implemented, and thus include at least one tangible unit capable of performing certain operations and may be configured or arranged in a certain manner. For example, a hardware-implemented module may comprise dedicated circuitry that is permanently configured (e.g., as a special-purpose processor, such as a field-programmable gate array (FPGA) or an application-specific integrated circuit (ASIC)) to perform certain operations. A hardware-implemented module may also comprise programmable circuitry (e.g., as encompassed within a general-purpose processor or other programmable processor) that is temporarily configured by software or firmware to perform certain operations. In some example embodiments, one or more computer systems (e.g., a standalone system, a client and/or server computer system, or a peer-to-peer computer system) or one or more processors may be configured by software (e.g., an application or application portion) as a hardware-implemented module that operates to perform certain operations as described herein.

Accordingly, the term “hardware-implemented module” encompasses a tangible entity, be that an entity that is physically constructed, permanently configured (e.g., hardwired), or temporarily configured (e.g., programmed) to operate in a certain manner and/or to perform certain operations described herein. Considering embodiments in which hardware-implemented modules are temporarily configured (e.g., programmed), each of the hardware-implemented modules need not be configured or instantiated at any one instance in time. For example, where the hardware-implemented modules comprise a general-purpose processor configured using software, the general-purpose processor may be configured as respective different hardware-implemented modules at different times. Software may accordingly configure the processor 1202, for example, to constitute a particular hardware-implemented module at one instance of time and to constitute a different hardware-implemented module at a different instance of time.

Hardware-implemented modules may provide information to, and/or receive information from, other hardware-implemented modules. Accordingly, the described hardware-implemented modules may be regarded as being communicatively coupled. Where multiple of such hardware-implemented modules exist contemporaneously, communications may be achieved through signal transmission (e.g., over appropriate circuits and buses) that connect the hardware-implemented modules. In embodiments in which multiple hardware-implemented modules are configured or instantiated at different times, communications between such hardware-implemented modules may be achieved, for example, through the storage and retrieval of information in memory structures to which the multiple hardware-implemented modules have access. For example, one hardware-implemented module may perform an operation, and may store the output of that operation in a memory device to which it is communicatively coupled. A further hardware-implemented module may then, at a later time, access the memory device to retrieve and process the stored output. Hardware-implemented modules may also initiate communications with input or output devices.

Computing systems or devices referenced herein may include desktop computers, laptops, tablets e-readers, personal digital assistants, smartphones, gaming devices, servers, and the like. The computing devices may access computer-readable media that include computer-readable storage media and data transmission media. In some embodiments, the computer-readable storage media are tangible storage devices that do not include a transitory propagating signal. Examples include memory such as primary memory, cache memory, and secondary memory (e.g., DVD) and other storage devices. The computer-readable storage media may have instructions recorded on them or may be encoded with computer-executable instructions or logic that implements aspects of the functionality described herein. The data transmission media may be used for transmitting data via transitory, propagating signals or carrier waves (e.g., electromagnetism) via a wired or wireless connection.

### Exemplary Logic and Supplemental Information

FIGS. 46-52D relate to “Exemplary Logic and Supplemental Information referenced herein, and support other figures of the Drawings.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A method of providing dynamically-alterable instructions to a patient, the method comprising:
extracting, by a processor, a plurality of responses to a plurality of questions associated with the patient;
conditioning, by the processor, a set of instructions based on the plurality of responses; and
updating, by the processor over a communication network, a dynamic document that contains a plurality of conditional fields whose value depend upon the plurality of responses thereby providing the set of instructions to the patient via the dynamic document.

2. The method of claim 1, wherein conditioning further comprises:
determining, by the processor, a first response to a first question;
determining, by the processor, a second response to a second question;
determining, by the processor, a third response to a third question; and
constructing, by the processor, the set of instructions based on the first response, the second response, and the third response, wherein the set of instructions contains a first line corresponding to the first response, a second line corresponding to the second response, and a third line corresponding to the third response, wherein the first line is dynamically updated based on the first response, wherein the second line is dynamically updated based on the second response, and wherein the third line is dynamically updated based on the third response.

3. The method of claim 2, wherein the first line is removed when the first response is changed to a fourth response of a different binary value than the first response.

4. The method of claim 3, wherein the fourth response is stored as a first binary value in a database, and wherein the first response is stored as a second binary value different from the first binary value in the database.

5. The method of claim 2, further comprising:
storing, by the processor, the first response, the second response, and the third response in a database, wherein the processor stores the first response as a first binary value, wherein the processor stores the second response as the first binary value, and wherein the processor stores the third response as a second binary value different than the first binary value.

6. The method of claim 5, further comprising:
determining, by the processor, a first logic combination, wherein the first logic combination is in a first state when the first response has the first binary value, the second response has the first binary value, and the third response has the second binary value; and
updating, by the processor and when the first logic combination is in the first state, the set of instructions to include a fourth line, wherein the fourth line provides further instruction to the patient.

7. The method of claim 6, further comprising:
determining, by the processor, the first logic combination, wherein the first logic combination is in a second state different from the first state; and
updating, by the processor, the set of instructions to remove the fourth line.

8. The method of claim 7, wherein the first question relates to a history of heart attack.

9. The method of claim 7, wherein the second question relates to the use of an anticoagulant.

10. The method of claim 7, wherein the third question relates to a type of insurance.

11. A device, comprising:
a communication interface that facilitates communication between the device and a medical provider system;
a processor coupled with the communication interface; and
a computer-readable storage medium coupled with the processor and comprising instructions that are executable by the processor, wherein the instructions comprise:
a set of instructions that provide a user of the device guidance on a plurality of pre-procedure actions.

12. The device of claim 11, wherein the processor:
determines a first response to a first question;
determines a second response to a second question;
determines a third response to a third question; and
constructs the set of instructions based on the first response, the second response, and the third response,
wherein the set of instructions contains a first line corresponding to the first response, a second line corresponding to the second response, and a third line corresponding to the third response, wherein the first line is dynamically updated based on the first response, wherein the second line is dynamically updated based on the second response, and wherein the third line is dynamically updated based on the third response.

13. The device of claim 12, wherein the processor:
removes the first line from the set of instructions when the first response is changed by the user to a fourth response with a different binary value than the first response.

14. The device of claim 13, wherein the processor:
stores in a database the fourth response as a first binary value, wherein the first response is stored in the database as a second binary value different from the first binary value.

15. The device of claim 12, wherein the processor further:
stores the first response, the second response, and the third response in a database, wherein the first response is stored as a first binary value, wherein the second response is stored as the first binary value, and wherein the third response is stored as a second binary value different from the first binary value.

16. The device of claim 15, wherein the processor further:
determines a first logic combination, wherein the first logic combination results in a first state when the first response has the first binary value, the second response has the first binary value, and the third response has the second binary value; and
updates the set of instructions to include a fourth line, wherein the fourth line provides further instruction to the user, and wherein the processor updates the set of instructions to include the fourth line when the first logic combination is in the first state.

17. The device of claim 16, wherein the processor further:
determines the first logic combination in a second state different from the first state; and
updates the set of instructions to remove the fourth line, wherein after the fourth line is removed, the user no longer receives any information from the fourth line.

18. The device of claim 17, wherein the first question relates to a history of heart attack.

19. The device of claim 17, wherein the second question relates to the use of an anticoagulant.

20. The device of claim 17, wherein the third question relates to a type of insurance.

21. The method of claim 1, wherein the method further comprises: populating, by the processor at a first time, the plurality of conditional fields of the dynamic document based on the plurality of responses.

22. The method of claim 21, wherein the method further comprises:
re-extracting, by the processor at a second time later than the first time, a second plurality of responses to the plurality of questions associated with the patient;
re-conditioning, by the processor at the second time, the set of instructions based on the second plurality of responses; and
updating, by the processor at the second time over the communication network, the dynamic document.

23. The method of claim 22, wherein updating the dynamic document at the second time includes changing a number of the plurality of conditional fields based on the second plurality of responses.

24. The method of claim 22, wherein updating the dynamic document at the second time includes presenting a number of additional instructions not present in the dynamic document at the first time.Z
